(19) United States
(12) Patent Application Publication
(10) Pub. No.: US 2024/0371522 A1
(43) Pub. Date: Nov. 7, 2024

(54) SYSTEM AND METHOD FOR DISEASE MANAGEMENT USING REINFORCEMENT LEARNING OR SYSTEM OF PHENOTYPES

(71) Applicant: Luminare Incorporated, Houston, TX (US)

(72) Inventors: Deepak Joseph, The Colony, TX (US); Narasimheswara Sarma Velamuri, Houston, TX (US); Marcus Joel Christian Persson Rydberg, Houston, TX (US)

(21) Appl. No.: 18/653,959

(22) Filed: May 2, 2024

Related U.S. Application Data
(60) Provisional application No. 63/463,373, filed on May 2, 2023.

(51) Int. Cl. G16H 50/20

(52) U.S. Cl. CPC G16H 50/20

(57) ABSTRACT

Disclosed is a system and method of detecting or assessing a medical or other health-related condition in a patient.

### FIELD

The present application claims the benefit of U.S. Provisional Application No. 63/463,373 filed on May 2, 2023 (pending), entitled “System and Method for Disease Detection Using Reinforcement Learning from Human Feedback (RLHF)”, the entirety of which is incorporated herein by reference.

The present disclosure relates generally to the field of disease management, including systems, methods, and apparatus for use in disease detection, including components, elements and/or stages/steps thereof, and further, computing systems and computing system implemented methods related thereto. More particularly, the present disclosure describes such exemplary systems and methods that employ artificial intelligence (AI) and/or reinforcement learning from human feedback (RLHF), and means and methods of generating or training such systems or AI models. Although systems and methods, and other aspects, disclosed are suitable for use in the detection or monitoring of various health conditions (e.g., of a patient), certain aspects are particularly suited for the detection or monitoring of sepsis as an example, as will be evident from the description and examples provided below.

### BACKGROUND

The systems and method described are particularly suited in the management of a patient that has or may be considered vulnerable to a disease or medical condition such as Sepsi (and similar conditions). In particular, sepsis is a life-threatening condition that ensues when the body responds to an infection and causes widespread inflammation. Early detection and treatment of sepsis are crucial to improving patient outcomes. Generally, detection of sepsis, in an accurate and timely fashion, is a complex and arduous task due to its intricate nature, dependence on several factors, and the limitations of existing knowledge and technology.

A sepsis screening system, screening tool or device, and method developed by the present Assignee of this disclosure is described in United States Patent Application Publication No. US20200234828A1 (“the '828 Publication”). The '828 Publication describes, among other things, an efficient method for assessing sepsis risk in patients. In one respect, the systems and methods according to the present disclosure provide additions and variations including enhancements and improvements to systems and methods, or elements or steps/stages thereof, that are well suited for addition to, or incorporation with, the system, screening tool and methods, including subprocesses and algorithms described in the '828 Pub. For example, the present disclosure teaches, an exemplary sepsis detection system and process that is realized by incorporating artificial intelligence and reinforcement learning from human feedback with concepts and embodiments introduced in that Publication. The '828 Publication can, therefore, serve well as background for the present disclosure and, accordingly, is hereby incorporated by reference in its entirety and for all purposes, and made a part of the present disclosure.

### SUMMARY

The present disclosure is generally directed to a system and method of detecting a medical or other health-related condition in a patient. Much of the description below specifically provides, however, an exemplary method and system for predicting, evaluating, or detecting sepsis risk in a patient and, more particularly, an exemplary system and method that incorporates a hospital protocol-based sepsis screening tool, electronic health records (EHRs), patient monitoring devices, an AI model and/or a system for generating a data set and further training the AI Model using RLHF. Concepts disclosed herein include processes, techniques, apparatus, structures, and devices, including components and steps therein and thereof, which are describe in the context of patient management and assessment of sepsis risk. Although, the monitoring or detection of sepsis is a primary example and provide an apt illustration of the application or embodiment of the disclosed concepts, the system, methods, and other aspect of the disclosure are also well suited for detection, monitoring, or management of other conditions. Accordingly, the disclosure presents concepts that are not limited to the specifically described systems or methods.

In another aspect, the present disclosure provides a system and method of managing and processing data pertaining to the presence or advancement of sepsis (or similar medical condition) in a patient. Alternatively, the present disclosure provides a system and method of learning and/or detecting the presence of sepsis in a patient using model-based reinforcement learning. In one embodiment, the system and method entail integration of reinforced learning from human feedback, and, preferably, in addition to supervised learning. As compared to traditional means, the systems and methods of the present disclosure achieve improvements in the accuracy and timeliness of assessment of the patient's condition, such as sepsis.

In one aspect, the present disclosure provides a system and/or method of patient or health condition management, and, as shown in described application, one well suited for management of sepsis risk. Further, the system or method described employs or incorporates an AI engine or module in patient or sepsis risk management. Relatedly, the disclosure provides a system or method that incorporates such an AI model or engine to build, modify, and/or enhance a system or method of sepsis detection. More specifically, an exemplary system and method of sepsis management, detection, or monitoring may employ or incorporate, in practice, reinforce learning human feedback. As well, an exemplary system and method of sepsis management, detection, or monitoring, according to the present disclosure, combines human expertise with machine learning and/or accounts for individual patient characteristics, and/or incorporates or adapts varying data [phenotypes] sources and integration methods.

In another aspect, a system and method is provided that generates or employs phenotypes (a system thereof) in assessing risk in a patient. In one application, a system of sepsis phenotypes is generated or employed. Further, a method of assessing sepsis risk includes generating said system of phenotypes, including deploying a model on or with a population of patient related data. A further method then includes assessing sepsis risk in a patient including using the model to assign a phenotype from the system of phenotypes.

For purposes of this disclosure, the term “assessment” or “assess” is used to refer to action taken and output provided, respectively, by a system processing patient-related data to predict, evaluate, or detect the presence of a medical condition and/or the degree to which that condition is present or pending. More specifically, and in this regard, the system or method is described also as assessing the risk to the patient of that condition being present or developing. Preferred methods of patient management described below entail the deployment of an AI Model to process or analyze data sets and assess, as output, the risk of Sepsis in the patient subject.

### BRIEF DESCRIPTION OF THE DRAWINGS

So that the manner in which the features and advantages of the present disclosure may be understood in more detail, a more particular description of concepts and aspects may be had by reference to the embodiments thereof which are illustrated in the appended drawings that form a part of this specification. It is to be noted, however, that the drawings illustrate only various exemplary embodiments and are therefore not to be considered limiting as to scope as it may include other effective embodiments as well.

FIG. 1 is a map provided by the CDC showing the disparity in sepsis mortality statistics across the United States;

FIG. 2 is a simplified diagram illustrating or representing an exemplary system and method by which an assessment of sepsis risk in a patient may be implemented, according to the present disclosure;

FIG. 3 is a simplified diagram showing the utilization of a screening tool in the system and method of FIG. 2, according to the present disclosure;

FIG. 4 is a simplified diagram showing exemplary stages in a process of handling data for use by a system and AI Model, according to the present disclosure;

FIG. 5 is a simplified diagram of a process of training an AI Model, according to the present disclosure;

FIG. 6 is a simplified diagram of a preferred subprocess for generating the AI Model in any one of FIGS. 2-5, as appropriate, utilizing Reinforcement Learning with Human Feedback, according to the present disclosure;

FIG. 7A is a simplified block diagram representing a relationship between an exemplary AI model and healthcare environment, according to the present disclosure;

FIG. 7B is a simplified process diagram of workflow within a healthcare environment implementing a system and AI Model, according to the present disclosure;

FIG. 8 is a simplified diagram representing an exemplary system and method by which assessment of sepsis risk in a patient may be implemented, shown configured for supervised learning and reinforced learning, according to the present disclosure;

FIG. 9 is a simplified diagrammatical flow chart illustrating a preferred process of generating phenotypes, according to the present disclosure;

FIG. 10 is a simplified diagram illustrating preferred subprocesses in a method of generating phenotypes, according to the present disclosure;

FIG. 11 is a plan view of a front display or face of a preferred embodiment of a Phenotype Match Card, according to the present disclosure; and

FIG. 12 is a flowchart representing a process of assessing sepsis risk that incorporates use of a system of phenotypes, according to yet another embodiment in the present disclosure.

### DETAILED DESCRIPTION

In one aspect, systems and methods employ a Machine Learning (ML) model(s) for a medical conditions that are well suited for integrating into the care and management of a patient and, thereby, collaborate in the patient's care. To this end, the system and method may contribute by generating system or caretaker notifications, in addition or including predicting or detecting the presence of a medical condition in the patient. As described in further detail below, preferred systems and methods deploy an AI Model to receive patient related information, analyze the inputted data sets, and output an assessment for the medical condition. In another aspect, the present disclosure describes a method of generating the system or, specifically, the preferred AI Model using supervised learning with training data sourced from past patient interactions and knowledge base. More preferably, a method of training an exemplary AI Model for assessing the risk of the medical condition, such as sepsis, entails employing reinforcement learning with feedback from responses of the admitted patient and/or human feedback (RLHF). The feedback in this case comes from interaction with the patient and/or attending medical personnel.

In a further aspect, the disclosed system and the models deployed are enhanced or adapted to mitigate the degradation of predictive performance over time. In yet another aspect, systems and method employ an AI Model that is adapted to account for concept drift—the change in the underlying data distribution or patterns over time. Preferred systems and methods account or consider new data or changes in patient demographics.

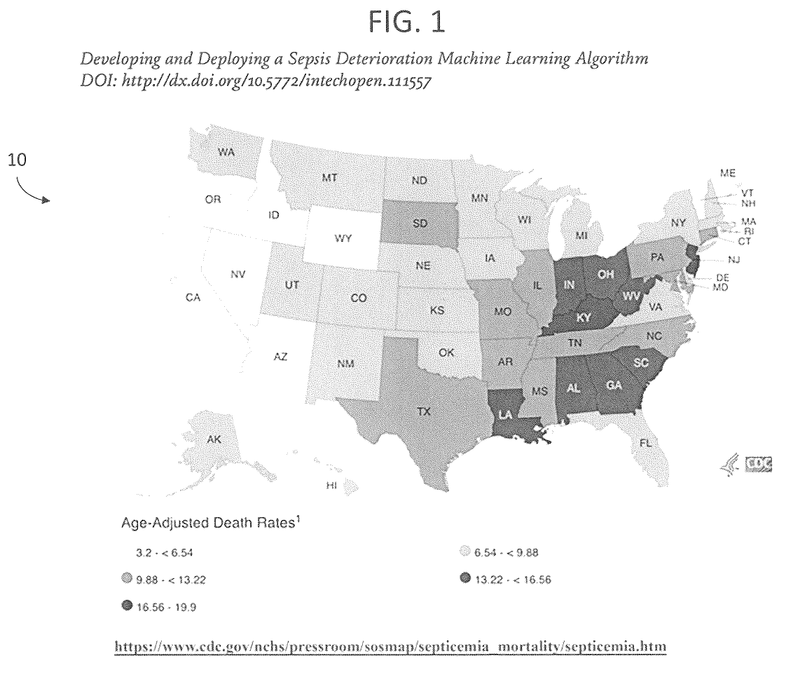

Preferred systems and methods recognize that an AI Model's predictive performance may degrade over time, particularly, when deployed on populations not resembling their original training sets. Accordingly, preferred embodiments of systems and methods described herein are designed to consider change in the underlying data distribution over time. Specifically, the systems and methods account, for example, new data or changes in patient demographics. To illustrate the present disclosure's contribution to the art, consider that sepsis death rates, or septicemia mortality, vary from state to state. See e.g., map 10 of FIG. 1, which is provided by the CDC at https://www.cdc.gov/nchs/pressroom/sosmap/septicemia_mortality/septicemia.htm. Models that are built in states with low death rates may perform poorly when deployed in states with high death rates and vice versa. This is due to the model overfitting to a particular population/dataset (i.e., one state versus another). Both data drift and concept drift can occur at the same time, leading to inaccurate predictions and reduced model efficacy.

It is preferred and advantageous to incorporate systems and methods that are adept at handling data drift, concept drift and population drift (collectively “drift”) in the maintenance and deployment of ML models. This is particularly impactful in the clinical setting, where predictions have an impact on patient outcomes. Accordingly, at least in one aspect and preferred implementation of systems and methods introduced herein, a means is provided for continuously incorporating prospective data or re-calibrating the model.

To illustrate further, a hospital may introduce new treatment protocols and acquire new patient monitoring devices. These changes may lead to subtle alterations in the patterns and types of data collected. For instance, a new device might measure certain vitals with increased precision or introduce entirely new metrics. Additionally, updated treatment protocols might lead to patients exhibiting different response patterns to sepsis. Initially trained on older data, the AI Model may begin experiencing decreased accuracy because of a lack of exposure to the new data distribution. This phenomenon is an instance of concept drift. The present systems and methods are adept at recognizing such drifts over time. By continuously integrating fresh data, retraining the model, and/or leveraging RLHF, the system adapts and recalibrates, ensuring consistently high detection accuracy despite evolving data landscapes.

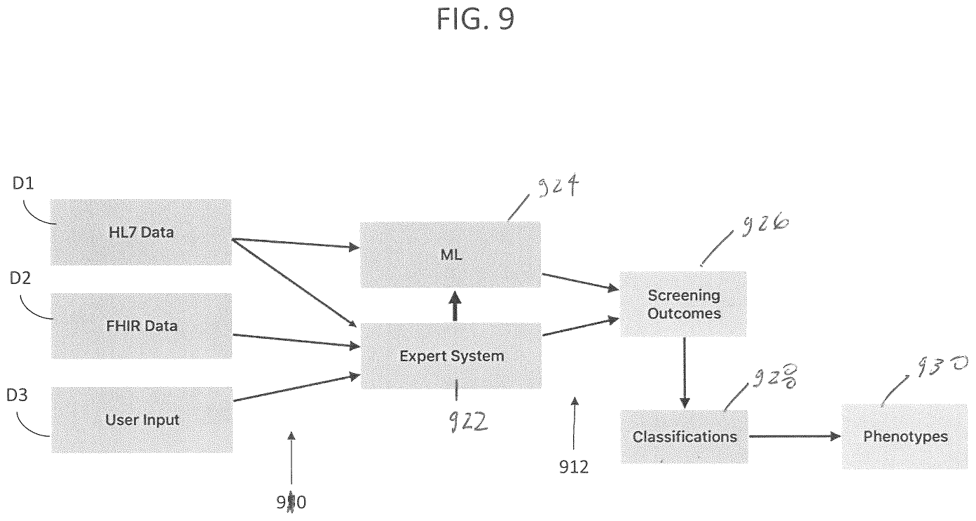

In another aspect, the present systems and methods leverage their capability and capacity to collect, access, and analyze large volumes of patient related data sets, including stored medical records, to generate new and intelligent classifications and/or relational subsets of data based on observable traits. These classifications or phenotypes are found to lend themselves well in the management of patients with a risk of a medical condition such as sepsis. In respect to much of the description and examples provided herein, core sepsis phenotypes and subtypes are developed. These phenotypes and subtypes capture the heterogeneous presentations of the disease across different patient populations. A method of generating phenotypes, their use in a method of assessing risk of a medical condition such as sepsis in a patient, including means for recalibrating or updating the method and corresponding system, and articles or products that embody or exemplify their usage are discussed in more detail below, particularly in descriptions accompanying FIGS. 9-12.

### Patient Screening Tool

In preferred systems and methods, a customizable sepsis screening tool is used by healthcare professionals and medical practitioners to evaluate patients for sepsis. A device suitable for applications according to the present disclosure is a sepsis screening tool that is described in the '828 Publication. The tool is customizable and can be used by nurses and clinicians to assess patients for sepsis. Preferably, the tool includes or executes rule-based algorithms to determine defined sepsis outcomes (SIRS, sepsis, severe sepsis, septic shock). The screening tool can be adapted for diverse patient populations and specific disease conditions, considering sundry factors such as age, medical history, and demographics. The screening tool produces an outcome which may indicate the presence or absence (or degree of confidence) of a given condition or the exigency for further evaluation predicated on pending lab results or other factors. As shown in the examples below, the screening tool provides a primary input data source in the deployment of the AI Model (to assess sepsis risk).

Preferably, the sepsis screening tool provided is congruous with a diverse array of EHR platforms and patient monitoring devices, thereby ensuring the seamless assimilation of data. The system is adept in accommodating various data formats and communication protocols to ensure compatibility with existing and future healthcare information systems. The versatility of the data integration process endows the system to adapt to the ever-evolving landscape of healthcare technology.

### Generating a System and Method of Assessing Medical Risk (Sepsis Example)

Generally, and most preferably, the presently disclosed system and method deploy an AI Model in the assessment of sepsis risk (including detection and monitoring) in a patient, which model uses Reinforced Learning Human Feedback, RHLF, tools to discover associations and formulas, and learn actions. The AI model preferably processes data received from input data sources such as a sepsis screening tool, EHRs, and/or patient monitoring devices to generate prompts or notifications for clinicians and caretakers, e.g., that a patient needs to be reevaluated. Once the patient is rescreened, the AI model uses the new screening outcome as feedback to learn and improve its sepsis detection capabilities. This RLHF approach allows the AI model to avoid overfitting and adapt to the heterogeneous nature of disease conditions and patient populations.

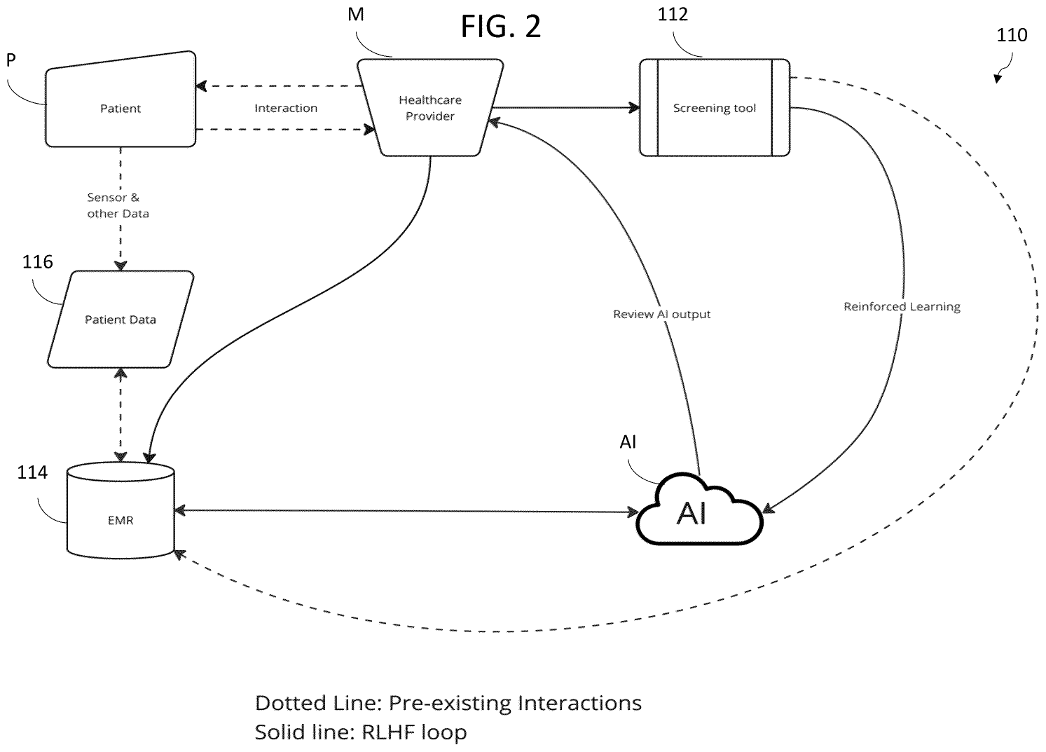

The diagram of FIG. 2 illustrates an exemplary system 110 (S) for assessing sepsis risk in a patient, highlighting certain beneficial aspects, and more particularly, the deployment of an AI Model to implement same. The diagram also represents a preferred method of assessing sepsis risk and illustrates the system and method of training the AI model using supervised learning as well as reinforced learning, with system feedback and/or human feedback.

The exemplary system 110 is drawn showing the elements and/or actors typically involved in the management of patient care, and, specifically, the assessment of sepsis risk in the patient. The patient P is received in a clinic environment and attended by a caregiver or medical professionals (collectively, healthcare provider, M). More particularly, FIG. 1 describes the interactions between the system S, patient P, and medical personnel M. In the diagram, dash lines signify interactions in traditional care settings, while solid lines signify additional interactions considered or integrated in the method of sepsis risk assessment, according to the present disclosure. In this exemplary system 100, a screening tool 112 is utilized by medical professionals (in interactions with the patient P) to receive patient data during the care timeframe. As shown, this patient data is (traditionally) communicated to a database 114 for storage. Typically, the database 114 is generally known as an Electronic Medical Records or Electronic Health Records database (simply, EMR or EHR) or equivalent. See e.g., the '828 Publication for further description. As well, patient monitoring devices may be physically disposed proximate the patient and provide and communicate additional patient data 116 to the EMR. Data may, therefore, be collected during initial interactions, such as in the Emergency Department (ER) or Hospital admissions (triage) or Inpatient care, or in later or continuing Inpatient care. Accordingly, the EMR may be accessed for patient data in assessing the patient for sepsis risk, and as further shown, in generating and/or training an advantageous AI Model.

Notably, the interactions between patient P and medical personnel M are two-way. An attending nurse or other medical professional may retrieve patient from the interaction, and also administer treatment or actions upon the patient.

In respect to the method for assessing sepsis risk according to the present disclosure, the system S includes and deploys an AI engine or Model to analyze patient data (past, present, and third party) and present, as output, a sepsis risk assessment (as further described herein in different subparts). The system 110 is configured such that the AI Model receives input patient data from the screening tool directly to and from the EMR. The AI Model then outputs and provides, medical personnel, a sepsis risk assessment based on the received patient data. In turn, medical personnel administer patient treatment and, as described below, may also provide feedback to the AI Model. Meanwhile, more patient data is received in the EMR, periodically, in real time, or in some combination, which changes and updates the data distribution analyzed by the AI Model. Through these interactions, the system 110 implements a reinforced learning with human feedback loop—for the assessment of sepsis risk in a patient. An exemplary loop is represented by the solid lines in FIG. 2.

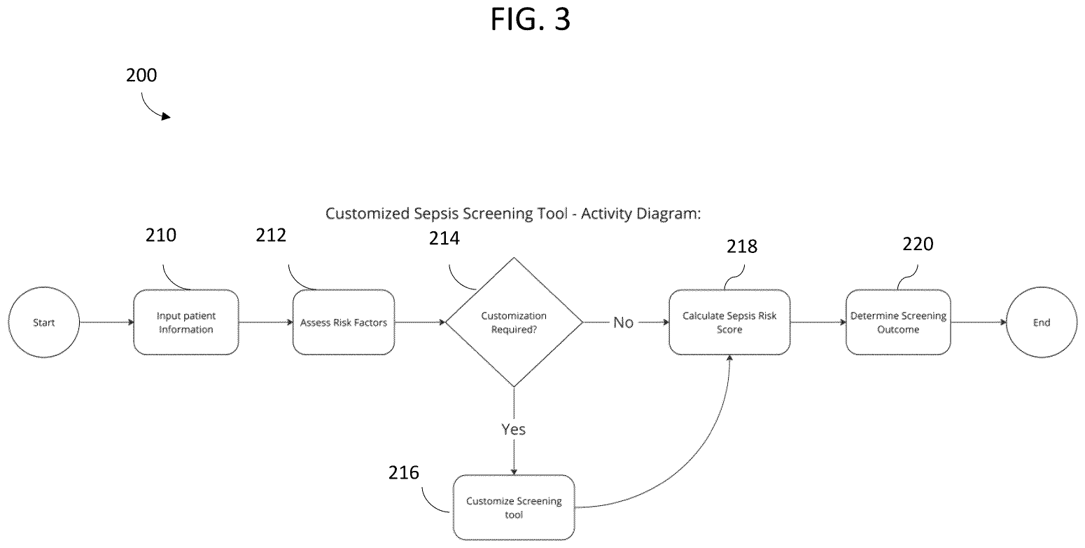

The diagram of FIG. 3 further illustrates the use of the screening tool 112 in the system 110 of FIG. 2, and in the accompanying method of sepsis risk assessment. In the interaction between patient P and medical personnel M, the screening tool is used to input patient information and convey that data to the system and AI Model (210). The system 110 then delivers an assessment of sepsis risk for the patient (212). Medical personnel or the system may evaluate the assessment as compared to actual observation and treatment (diagnosis), and determine whether the screening tool requires customization to capture any insight gained (214). Thereafter, the screening tool may be customized, or adjusted, so as to reflect the added insight or changes (216). For example, new queries may be incorporated. In patients who have heart transplants and undergo vagal nerve denervation, for example, heart rate and blood pressure thresholds may be changed and the screening tool functionality may be modified to incorporate monitoring of additional factors such as lactate dehydrogenase (LDH-different from lactic acid level) and albumin levels. This type of adjustment works to increase the AI Model's precision and recall. Notably, customization of the screening tool may be effected automatically (immediately used in subsequent screenings and assessment), by the system (or, by medical professional in some instances).

Returning to FIG. 3, the system delivers a sepsis risk score (218) to medical personnel and the new results and screening outcome may then be evaluated (220). The new results and screening outcome is displayed to a user, preferably as a clinical phenotype as described below, and may be evaluated by medical personnel M. The screening tool is particularly suited to this as the various un-explainable parameters that were used in the decision of the AI model can be reflected back to the user in a meaningful context (e.g. LDH and albumin, were low in this patient with heart transplant who have a higher chance of developing sepsis than those with normal levels).

### Training the AI Model—Supervised Learning

Accordingly, a preferred system may be generated utilizing an AI model, as further described herein, which may entail training the AI model based on selected data sets. The preferred AI model is a machine learning model designed to process and analyze data received from an array of inputs. For present purposes of illustration, the AI model may be configured to generate, as output, from patterns found in labeled data, the presence of, or prediction of, the sepsis in a subject patient, or a decision on treatment. The output may include a confidence score or degree of urgency or advancement. Herein, such output may be referred to, generally, as an assessment. In a healthcare, clinic, or academic environment, the data sets source(s) may be served by Electronic Medical Records (EMR) established from historical patient (interaction) data. The data sets may include patient condition information, including vitals, past assessments, treatment or actions taken, further results and outcomes, and other general knowledge.

Thus, in an exemplary, initial phase of AI model training, supervised learning is employed using labeled data from historical sepsis cases. The data may comprise patient information, sepsis screening outcomes, and relevant clinical data. Specifically, the AI model is preferably trained to identify patterns and relationships between the input features and the presence or absence of sepsis. In this respect, various machine learning algorithms, such as decision trees, support vector machines, and/or deep learning models, may be used depending on the data characteristics and the desired level of model complexity.

In accordance with a method or application of the present disclosure, the AI model is integrated into the system application and then deployed to assess sepsis risk in a patient admitted and introduced into the system. Data input sources preferably include a sepsis screening tool or device, as described previously. The device may be a handheld tool operable by medical personnel attending to a patient who is presented for evaluation and observation. Patient monitoring devices may also be configured to receive physical data from the patient (e.g., heart rate, temperature, respiratory rate, peripheral capillary oxygen tension/saturation, blood glucose levels, etc.). These data values may be inputted into the screening device by attending personnel and linked and uploaded onto the system network. As also described in the '828 Publication, patient screening may be augmented by an array of patient monitoring devices. These include blood pressure machines, Electrocardiograms, pulse oximetry, invasive oximetry, invasive blood pressure monitoring, or any other real-time patient monitoring input. Other data sources, such as information received from wearables, medical imaging, or genomics data may also provide input, and in real-time or after initial assessment and treatment. The AI Model is thereby trained with these input data sets and the historical labeled data (discussed above).

Further, a preferred system and AI Model may be trained on large volumes of data, of possibly disparate types, character, and formats, to generate phenotypes. This is discussed in more detail below. It will become apparent to one skilled in the art, however, with access to the present disclosure, how the systems, methods, subprocesses, and techniques described in respect to FIGS. 2-8 may be applicable to such preferred systems and methods relating to or utilizing phenotypes (or systems of phenotypes). Although the systems and methods are not limited to use or incorporation of a system of phenotypes classification and the like, reference may be made below to such use or incorporation in a manner that illustrates the advantageous aspects and the implementation of the described concepts and embodiments.

In generating or training the AI Model, the present system and method preferably employ system-based feedback, which entails one mode for continual adjustment of the Model's algorithms based on outcomes. For example, the Model may find certain patterns of vital signs, laboratory results, and patient characteristics which more frequently result in sepsis. Accordingly, the Model may adjust its algorithms to recognize relevant patterns and, thereafter, provide more accurate predictions. The Model may also find that the type of sepsis presentation (e.g., index phenotype) that the patient presents with and their clinical progression (e.g., secondary phenotype) determines what medications to administer to the patient (e.g., patients who present with altered mental status should not be given synthetic penicillin containing antibiotics). (See also discussions below on phenotypes particularly in respect to FIGS. 9-12.

Unlike many other conditions, sepsis is not a single disease but a syndrome resulting from a dysregulated host response to an infection. Moreover, the patient may be subject to varying sources of infection, and indicate different immune response intensities and diverse comorbidities. For instance, a patient with pneumonia and diabetes may develop sepsis differently from a patient with a urinary tract infection and heart disease. Thus, sepsis can manifest differently among patients. The Model will ensure that a correct risk score is associated with each of these types of patients. For example, in the above referenced case of a patient with a urinary tract infection and heart disease, consider that the patient's heart disease causes heart failure and subsequently lower resting blood pressures. The patient may be on medications that do not allow the patient to develop a tachycardia based on the patient's phenotype. The deep learning system will establish a new baseline blood pressure and heart rate for such patients and be able to more accurately predict when the patient decompensates from worsening urinary tract infection that develops into sepsis. In accordance with the present disclosure, systems and methods are provided to account such cases and possibilities, establish AI models, and improve on the reliability and accuracy of such models.

Further, the preferred systems are well suited for adaptation to “individual patient characteristics” and “personalized risk assessment.” Generally, if a target for a disease condition is set to train an Artificial Intelligence (AI) system, the expectation is that surrogate markers of the condition are to be used to define whether the disease is present or absent (positive or negative). For sepsis detection, AI DRG codes and ICD-10 codes for sepsis may be utilized, or natural language processing is utilized, to extract free text from unstructured data. This approach may cause low specificity and alert fatigue because the target being defined is binary, i.e. that sepsis is either present or absent. This methodology may cause further problems because often, in clinical practice, what is deemed as sepsis from a model is not agreed upon at bedside by providers. Accordingly, preferred systems and methods classify sepsis into seven separate phenotypes-5 adult, 1 NICU phenotype (with several subgroups), 1 Pediatric phenotype (with several age based further subgroups); additional phenotypes have been identified in populations with neoplasms, burns and so on. The neonatal sepsis and pediatric subgroups can be expanded into further categories as more information is available from newer sepsis biomarkers and new sensors like real-time lactate monitoring and others are deployed clinically.

The system facilitates personalized sepsis risk assessments by comprehensively analyzing each patient's unique medical history, current clinical state, and existing comorbidities. For instance, the system might classify a patient with a minor infection as “high risk” due to concurrent factors such as diabetes and compromised immune function. This assessment is further substantiated when non-traditional symptoms—such as changes in mental status rather than typical signs like fever or increased heart rate—are present. These symptoms are indicative of a phenotype associated with more severe outcomes and prolonged intensive care unit stays. This nuanced understanding not only aids in precise risk stratification but also enriches the system's knowledge base.

Each patient interaction enhances the AI model's training, turning individual cases into a repository of insights that inform future assessments. For example, an initial dataset comprising 500 liver transplant patients with a specific phenotype can be significantly expanded through real-world deployments in transplant centers. This expansion not only broadens the dataset but also deepens the system's understanding of complex phenotypes, thereby improving predictive accuracy and patient care outcomes.

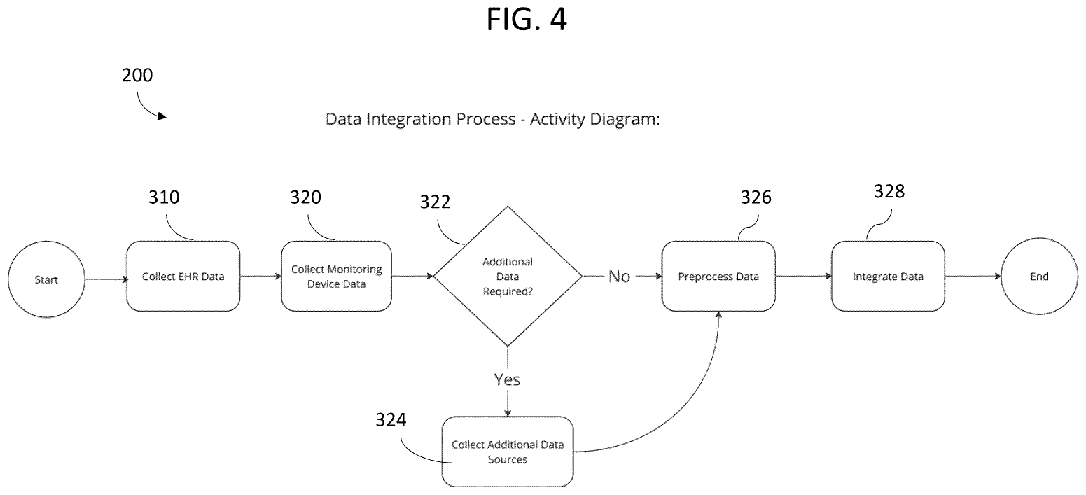

FIG. 4 illustrates a process of gathering data for use with the AI Model in FIG. 2, including training the AI Model. The development of the AI Model begins with the collection of data—from available patient or sepsis-related data in the present example. As discussed above, EHR databases may be accessed (310)). This data volume may also include patient data collected by the healthcare provider, and, during, deployment of the methods and AI Model, additional data may be collected from patient monitoring devices (320). In establishing the initial data distributions for training the AI Model, the system or healthcare entity (or other actor) may choose to collect additional data sources, including additional data form their own patient interaction over time (324). The decision to collect or not collect data (322) may be a matter of design choice, or more importantly, directly tied to the degree of confidence or responsibility entrusted to AI Model at the initial stages. As shown in this disclosure, the performance of the AI Model is expected to improve with time, with more data, and with employment of refinement techniques discussed in this disclosure.

Data collected may be preprocessed (326) prior to being of optimal use to the AI Model. Pre-processing may be performed by an expert system (of algorithms) that, in further embodiments, is the same expert system that clinical users use to evaluate sepsis phenotypes (i.e. via the screening tool). The data can then be integrated in or with the AI Model (328), and further, with the data distribution or arrangement already established for all AI Model data. This proves beneficial, as the expert system is able to perform feature extraction to the exact parameters that are determined from the user's previous interactions with the system (as in the above example of a patient with a heart transplant).

### Training the AI Model—Reinforcement Learning

Once the initial supervised learning phase is completed, the AI model preferably transitions to a reinforcement learning phase. More preferably, the AI Model is trained using reinforcement learning with human feedback (RLHF) methodologies to augment its proficiency in identifying sepsis, while also progressively refining its knowledge base. In this phase, the AI model may process data received from input data sources such as a sepsis screening tool, EHRs, and/or patient monitoring devices to generate prompts for clinicians and caretakers (sepsis risk assessment). The system prompt may alert medical personnel or caregiver to reevaluate the patient, as appropriate, and use the screening tool to rescreen the patient. The AI model then uses the new screening outcome as feedback to learn and improve its sepsis detection capabilities. In this further training phase, the AI model uses this feedback to update and refine its understanding of sepsis detection, allowing it to adapt to the heterogeneous nature of disease conditions and patient population.

In the course of patient management, the system and method employ reinforcement learning algorithms in its model to account for information received through human feedback. These algorithms allow the model to learn and evolve with each new case. For instance, the Deep Learning algorithm may be used to optimize the prediction of sepsis onset by continually improving the prediction model based on the reward mechanism. The “reward” here is the successful early prediction of sepsis, enabling timely intervention and better patient outcomes. In further applications, a degree of success or score may be given based on treatment outcome, accuracy, and/or completeness of prediction.

Feedback enhances the AI model ability to learn and refine its predictive abilities. Human-based feedback from medical professionals in charge of the patients care, or other qualified authorities, may include a validation or questioning of the model's predictions, or a communication of actual patient outcome(s) or additional patient data. This type of feedback is used in refining the model's analysis and assessment of sepsis risk. To illustrate, the AI model may flag or assess a patient as “high risk” for sepsis and prompt medical personnel accordingly. In this example, the attending clinician disagrees with the assessment, which the system records, and, perhaps, with further observation, contradictory assessment, and/or treatment recommendations. The patient then continues on a course that does not lead to sepsis development. This set of information (the initial assessment, the clinician's agreement/disagreement, timeline, the patient outcome, etc.) provides feedback for the AI model. Accordingly, the AI model preferably adjusts its algorithms to reduce the likelihood of similar false positives in the future and/or capture data sets material to the predictive analysis, thereby improving the AI model's future performance. In another example, the clinician agrees with the system alert, takes preventive actions, and the patient does not develop sepsis. This is also informative feedback for the model and is used in reinforcing the validity of similar predictions. In further extensions of these examples, the AI model also receives such data as patient vitals over associated time periods, markers indicative of the degree or timing of sepsis and other symptoms, interventions taken, other changes in the patient care and in the care environment, and these are accounted for in the AI model's analyses.

The exemplary system and method of sepsis risk assessment are, therefore, well suited for use in the management of a patient admitted to a hospital or healthcare system. The patient's existing patient records may be accessed via the EMR but, in the clinic setting, additional patient data is retrieved through use of patient monitoring devices and/or “bedside” observations (e.g., inputted via the screening tool mentioned above). In this example, the patient is diagnosed with hepatocellular carcinoma at the clinic, and the system is provided information that the patient is exhibiting signs of pyrexia, tachycardia, and tachypnea, which is possibly indicative of sepsis or bacterial infection (recognized as such by the exemplary system). In further accordance with, and so as to evaluate the patient, the nurse employs the sepsis screening tool and verifies that no opioids are being administered. While the AI system initially identifies a possible instance of sepsis based on the data collected, the healthcare professional concludes that the patient's symptoms are actually caused by inflammation due to hepatic carcinoma rather than sepsis. The clinician then proceeds to inform the AI model that the alert was a false positive.

In this application of the system or method, the AI model learns from human feedback and updates its understanding of the patient's condition. Employing this knowledge, the AI disentangles sepsis from inflammation instigated by malignancy or other afflictions such as solid tumors, hepatocellular carcinoma, sarcomas, leukemias, or end-stage heart failure with new stage 3 chronic kidney disease. Such astute discernment guarantees that patients receive befitting therapy and attention for their particular condition.

In the example above, the method employs the system and the AI model, to aid clinicians in evaluating patients (more precisely) diagnosed with solid neoplasms who display comparable symptoms. As an illustration, the system with AI model notifies the clinician that the fever and tachycardia are a result of the tumor, while the tachypnea is instigated by the pain. The AI model may suggest the administration of acetaminophen or opioids and carry out a subsequent evaluation in four hours. Such a personalized notification assists healthcare professionals in providing suitable interventions and evaluations, which can ultimately result in superior patient outcomes.

This example illustrates how the sepsis detection system, through the integration of human feedback and AI learning, adapts to different patient conditions and provide more accurate assessments and recommendations for interventions. FIGS. 4-8, and accompanying descriptions, provide further illustration of the exemplary systems or methods and how these may be advantageously configured or implemented.

### Further Enhancements/Programs

### Model Evaluation and Validation

The performance of the AI model is evaluated using standard metrics, such as sensitivity, specificity, and area under the receiver operating characteristic curve (AUROC) and/or area under precision recall curve (AUPRC). To ensure the model's reliability and generalizability, cross-validation techniques, such as k-fold cross-validation or stratified sampling, can be employed during the training and validation process.

### Model Explainability

To foster trust in the AI model's predictions and improve its adoption by healthcare professionals, model explainability techniques may be incorporated in exemplary systems and methods. Explainability techniques such as Local Interpretable Model-agnostic Explanations (LIME), Shapley Additive Explanations (SHAP), an feature importance analysis are among a number of suitable tools. Implementation of these techniques help elucidate the reasoning behind the model's predictions and the contributions of different factors to the sepsis risk assessment. As the system discovers correlations between the features of patients and the particular phenotype to which they belong, these correlations may indicate additional factors that would then be extracted to add additional functionality to the system as a whole. This explainability would be leveraged to improve existing phenotypes and possibly to create entirely new groupings of patient classes. See further discussions below.

### Exemplary AI Model Algorithms

An exemplary sepsis detection system employs an AI model leveraging the power reinforcement learning and boosting algorithms to reduce bias and variance in supervised learning. Such algorithms consist of models that make very few assumptions about the data with respect to a distribution and adding them to a final strong classifier. Notably, the solution makes use of gradient boosting (through the CatBoost framework) as well as the Actor Critic Method, Deep Deterministic Policy Gradient (DDPG), Deep Q-Learning and/or Proximal Policy Optimization. The following section detail unique examples of how these algorithms are utilized.

### Actor Critic Method

Incorporating the Actor Critic Method into an exemplary method or system allows the system to learn the optimal policy using two neural networks: the Actor network, which is responsible for taking actions, and the Critic network, which evaluates those actions. In the context of sepsis detection, the Actor network might determine whether a particular patient symptom or lab result indicates a high risk of sepsis. The Critic network then provides feedback, allowing the Actor network to improve its future decision-making. For instance, in the case of a patient with persistent fever and an identified source of infection, the Actor network might initially overlook the likelihood of sepsis. However, the Critic network, armed with feedback from healthcare professionals and patient outcomes, corrects the Actor's judgments, thereby improving future sepsis detection. As the patient could have developed a secondary source of infection. Further existing sepsis alert systems are fraught with false-positives and create enormous alert fatigue. Present methods allow for the critic—the bedside clinical team—to granularly indicate if a particular prediction was false—by, (a) not enacting the sepsis workflow that the system is expecting, and (b) proactively indicating why that particular patient did not have a sepsis phenotype e.g., patient is post coronary-artery-bypass surgery and fever is common for the first 48 hours post-surgery.

### Deep Deterministic Policy Gradient (DDPG)

As preferably implemented, a system and method utilizing DDPG techniques allow the AI model to handle continuous action spaces, which can be particularly useful in sepsis detection given the multifaceted nature of the condition. For instance, a patient's clinical indicators-temperature, heart rate, respiratory rate, and others-exist on a continuous spectrum. Using DDPG, the AI model can effectively learn from these continuous variables to predict sepsis risk. The learned policy can identify subtle changes in the patient's vitals, which may be indicative of the onset of sepsis, allowing early intervention.

By applying DDPG techniques, the system can leverage continuous action decision-making capabilities to fine-tune treatment strategies for individual patients, potentially leading to more personalized and effective care.” For example, today there is little to no understanding on the impact of different antibiotics on mortality. New literature points towards certain classes of antibiotics potentially increasing mortality and other classes of antibiotics decreasing mortality. Another example is the type of crystalloid fluid administered in septic shock could either increase or decrease mortality. In the present case, the AI would be able to inform a nurse or physician at bedside on the optimal antibiotic and the optimal amount and type of crystalloid fluid to administer to the patient.

Based on boosting in a functional space, gradient boosting focuses on a space where the target is pseudo-residuals rather than the typical residuals used in traditional boosting. A prediction model is provided in the form of an ensemble of predication models that make very few assumptions about the data. The solution leverages a gradient boosting framework (CatBoost) which, among other features, attempts to solve for categorical features using a permutation driven alternative to the classical algorithm.

With gradient boosting the algorithm begins by making a initial guess at one or more target variables. Gradually, it constructs an ensemble of decision trees, each tree aiming to reduce the residuals from the previous trees.

### Deep Q-Learning

While developed for gaming scenarios, Deep Q-Learning is employed in exemplary systems and methods for sepsis detection. The algorithm involves the AI learning to optimize its actions based on rewards—i.e., accurate sepsis detection and timely intervention. For instance, considering an infection scenario as the “game”, the AI model learns to “play.” effectively by accurately identifying sepsis from various indicators (actions) and improving patient outcomes (rewards). Over time, the AI model becomes increasingly proficient at “winning the game,” i.e., detecting sepsis early and accurately. For example, the Q in Deep Q-Learning is the estimated rewards from taking specific actions and in the sepsis use case the reward would be a “real” action like ordering a lactic acid blood test, two sets of blood cultures, within 60 minutes of the system identifying a patient has having potential sepsis. When the corresponding clinical action takes place, the system is rewarded.

### Proximal Policy Optimization:

In alternative systems and methods, a Proximal Policy Optimization (PPO) reinforcement learning method is employed. With this method, the model seeks a balance between exploration (of uncharted policies) and exploitation (of known policies). Incorporated in the preferred systems and methods, PPO is employed to continuously learn and adapt to new patterns of sepsis presentation while still effectively recognizing and acting upon established sepsis indicators. For instance, if a new strain of bacteria emerges causing sepsis with slightly different symptoms, the PPO algorithm ensures the AI model can explore this new “policy” of sepsis identification without completely discarding its existing knowledge base. In another example, if a patient is younger than 41 years old and has rapidly deteriorating septic shock with no antecedent clinical complaints and is noted to have isolated limb swelling, the AI Model would recommend considering exploratory faciotomy on the swollen limb to exclude necrotizing fasciitis as the underlying cause of sepsis.

### Parameters Interaction and Change Over Time

In another, advantageous aspect of systems and methods of sepsis assessment according to the present disclosure, the AI model employed or incorporated is adapted to account for and analyze relation or interaction between parameters (possible sepsis factors) and/or the change in data values over time (function over time). The system attempts to account for Sepsis that may manifest through numerous bio-physiological parameters and that these may evolve over time. These parameters may include heart rate, blood pressure, body temperature, respiratory rate, or blood count. Also, the system capabilities described herein allow the system to accommodate input of other data, including patient or treatment specific data that may not be considered in traditional assessments. The system and AI model is configured to account if and how these bio-physical parameters are interconnected and how these parameters function relatedly, which takes the form of a web of causative and associative factors.

To illustrate, an admitted patient may be found with an initial presentation of high fever due to a bacterial infection. The immune response to the infection might increase the heart rate and cause a drop in blood pressure. The fever, heart rate, and blood pressure are input data that may be monitored and fed into the AI Model in real time. As sepsis progresses, the fever might subside due to the use of antipyretics, while the heart rate could remain elevated due to increased systemic inflammation. Additionally, the patient's blood work might show an elevated white blood cell count, indicating an active fight against infection. However, this blood cell count could drop as sepsis worsens and immune system exhaustion sets in. So, in this example the AI model is able to assign a phenotype to this patient into a category of worsening severe sepsis and alert the clinical team to escalate antibiotic therapy or look for an untreated source of infection or a fresh source of infection. In view of such dynamic changes and complex interactions, an AI model is disclosed that can integrate diverse data, discern patterns, and anticipate disease progression in real-time. By providing a large and vetted corpus of data on past patients as training data (as discussed earlier), an AI model is generated that can draw a variety of inferences between the data in the training set and novel situations observed in the environment. These inferences are then used to ensure that the incoming data is used to appropriately indicate the patient's membership in a particular clinical phenotype.

To further illustrate, over the course of a year, the hospital introduces new treatment protocols and acquires new patient monitoring devices. These changes lead to subtle alterations in the patterns and types of data collected by the system. For instance, a new device might measure certain vitals with increased precision or introduce entirely new metrics. Additionally, updated treatment protocols might lead to patients exhibiting different response patterns to sepsis. In preferred applications, the AI model, initially trained on older data, may experience a decrease in accuracy-due to lack of exposure to the new data distribution. This phenomenon is an instance of concept drift. For example, after a major hurricane hit the Louisiana coast a large population of residents moved to the Houston metropolitan region. They carried with them the microbiome, resistance pattern and changed the demographics of the patients appearing in the emergency department in the world's largest medical center—the Texas Medical Center in Houston. Any AI model that had been trained prior to this large population shift would immediately be ineffective due to concept drift. The system and method employ programs to detect such drifts over time (and correlation to new information). By continuously integrating fresh data, retraining the model, and leveraging RLHF, the system can adapt and recalibrate, ensuring consistently high detection accuracy despite evolving data landscapes.

In one respect, systems and methods described herein are particularly suited for predicting, diagnosing, modeling, and/or managing the treatment or diagnosis of a medical emergency such as sepsis, as presently described, given its accuracy and speed of performance. Typically, the sooner that sepsis is detected and treated, the better the outcomes. A system or method employing, or in accordance with, an AI model for sepsis prediction, according to the present disclosure, is equipped to make real-time predictions-a complex task considering the number of variables that is integrated and rapid rate of the progress (as compared to cancer, for example).

Furthermore, the systems and methods described herein are particularly suited for use in the context of sepsis prediction and treatment because of its AI model's capacity to incorporate the continually evolving medical community's understanding of sepsis and related research. An AI model for sepsis detection, as provided herein, is adaptable to incorporate the latest research findings and reflect changes in clinical guidelines. Reference may be made for example, to sepsis guidelines provided by The Center for Medicare & Medicaid Services, which provides sepsis guideline updates on a quarterly basis (for Medicare reimbursements) or to private (insurance) payors that provided their own criteria for reimbursements.

In accordance with certain exemplary embodiments, the system or method described incorporates an AI model with an integrated, customizable alert system. This provides the ability to notify clinicians of different levels of sepsis risk, helping them prioritize patients in need. For example, a “high-risk” alert could initiate immediate medical review, while a “moderate-risk” alert could result in more frequent patient monitoring.

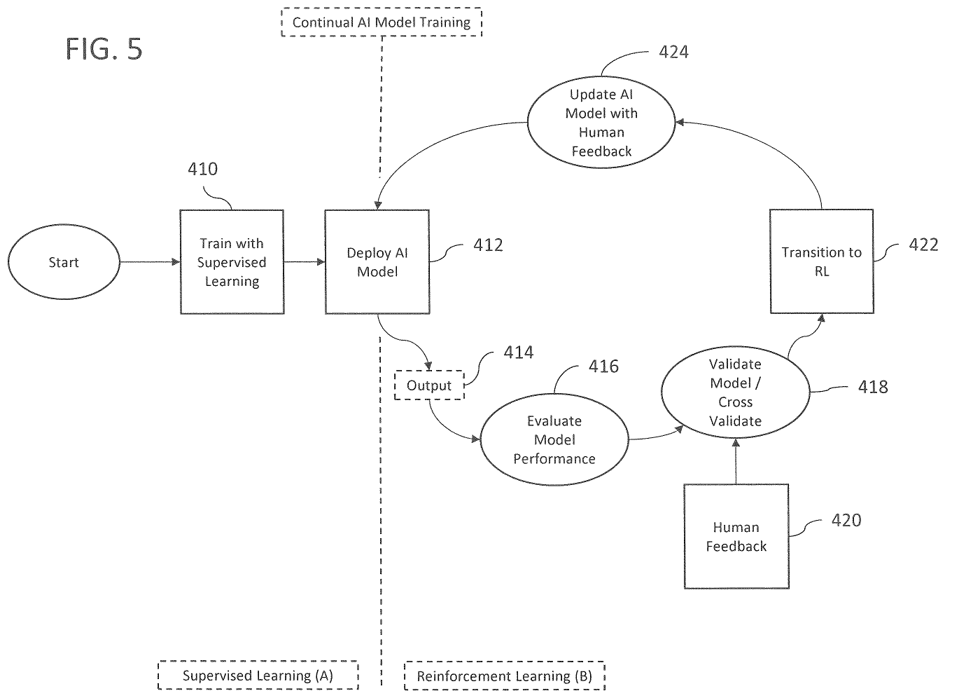

The simplified diagram of FIG. 5 summarizes the above-described preferred method or process of training the AI Model (400), which may be described in two stages or phases-Supervised Learning (A) and Reinforcement Learning (B). Initially, a system is provided that comprises a computing system configured with processing and storage capabilities, and containing an AI Model or engine. The computing system also supports and executes various programs and routines that effect subprocesses in the method of assessing sepsis risk, deployment of the AI Model, or in the training or refinement of the AI model. In a preferred first stage 410, the AI model is trained under supervised learning using labeled data derived from historical sepsis cases. Such data may be retrieved from an EMR database, for example.

After the AI model 412 is deployed and outputs a sepsis assessment(s) 414, the Model performance is evaluated, in a subsequent stage 416. The performance of the model is then evaluated in two different ways. Notably, this evaluation data is collected, stored, and then used in subsequent fine-tuning processes to continuously improve the performance of the model without endangering the model's fitness for use in a variety of clinical settings. Firstly, the system itself allows for the collection of subjective feedback from a clinician. Preferably, this feedback includes an indication of whether the clinician decided that additional clinical measures needed to be enacted. The clinician's decision may be based on a phenotype classification provided by the model. This information is collected from the clinician while they are in the presence of the patient and immediately after presentation of the patient's classification. Secondly the system monitors a variety of data provided by an electronic health record system (EHR) for indications that specific types of treatment have been administered to the patient subsequent to phenotype classification. As data from the EHR system arrives, and once a threshold of indicators have been recognized, the system will differentiate between those patients who received treatment in accordance with a particular phenotype assignment (agreement) and those who did not (disagreement).

This is followed by a validation stage 418, which may entail employment of cross-validation techniques. AI model training continues and is further enhanced (and updated) through human feedback 420. As discussed above, human feedback may come from interactions with medical personnel who agree or disagree with the model's predictions and an accounting of the actual outcomes. At this point, training transitions to Reinforcement Learning (422) and then, the AI Model is updated (424) accordingly. The improved AI Model is then deployed in assessing the risk of sepsis in a patient. In practice, and as shown by FIG. 4, reinforcement learning continues and the AI Model is continually evaluated, refined and updated.

In respect to a preferred method (and system) that entail generation and use of phenotypes, as discussed further below, the type of validation used will depend on the incidence of that particular phenotype in the population. For example, one of an AUROC technique or AUPRC technique may produce more advantageous results. These metrics, focusing on the performance concerning the minority class (sepsis is present), provide a clearer insight into a model's real-world utility in predicting critical but infrequent conditions of rarer sepsis phenotypes. Training then moves to reinforcement training which entails integration into nursing and clinical workflow and having feedback from the bedside team on the accuracy of the AI predicted phenotype and if not accurate the reason for the false positive. In this respect, see above example wherein hepatocellular carcinoma is described as causing a false positive and, a medical professional (nurse) submitted feedback to the system on the reason for a false positive

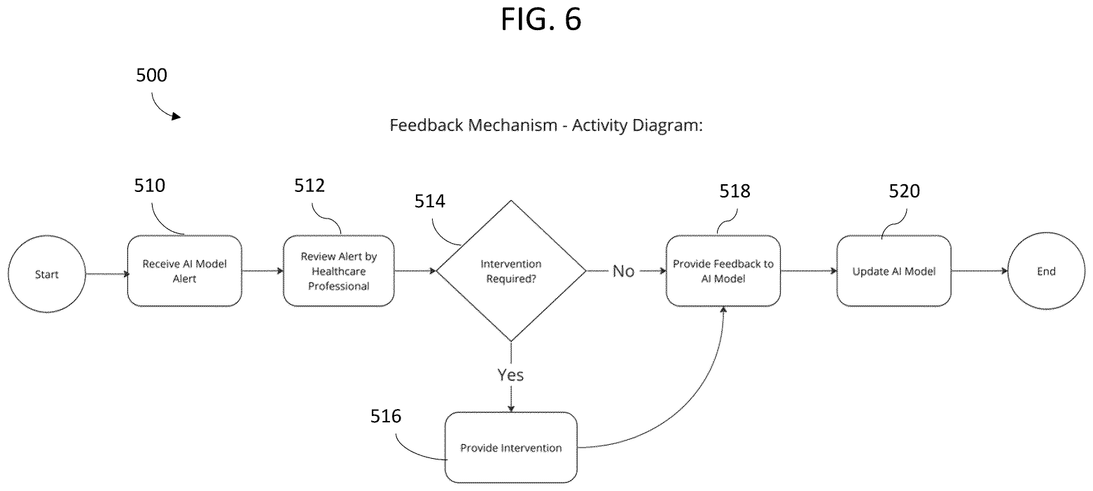

FIG. 6 illustrates the steps or stages in an exemplary method of training the AI Model with Reinforcement Learning with Human Feedback (500). The process diagrammed provides a snapshot in the continual training of the AI model. In this example, the clinic or medical environment receives sepsis risk assessment from the AI model (or system), which may be based on analysis of actual patient data as well as historical data. In any event, the AI Model initiates an alert (e.g., high risk of sepsis) that is received by medical personnel (510). The attending medical professional reviews the alert (the AI Model's output) (512). At this junction, the medical professional decides, based on his/her professional judgement and/or other information available, whether to agree or disagree with the assessment (514). If the medical professional disagrees, then intervention is required (“Yes”) and the professional provides intervention (516). The fact of intervention is feedback to the AI Model (518). If the medical professional agrees, then no intervention is required (“NO”), and this fact is also communicated as feedback to the AI Model (518). This sequence or instance then becomes part of the data distribution considered and analyzed by the AI Model to make risk assessments, thereby updating the AI Model 520. In actual or more extended applications, the “intervention” data communicated to the AI Model will go beyond the Medical Professional's agreement or disagreement. Information on action taken by the clinicians and the patient's response or outcomes provide valuable feedback that help improve the AI Model's performance.

### Virtual Nurses

In a preferred embodiment, remote virtual nursing is incorporated into the sepsis detection and intervention system and method. This enhancement transforms the traditional on-site nursing paradigm, providing a scalable solution that alleviates workload burdens, increases patient care efficiency, and significantly enhances sepsis detection and intervention processes. Remote virtual nurses are integrated into the system to conduct proactive sepsis screening for all patients in the Emergency or inpatient unit at the beginning of each shift. Using a sepsis screening tool, as discussed above, and real-time patient data, virtual nurses assess sepsis risk and adapt the screening process based on individual patient characteristics and the evolving nature of their condition. Further, the preferred AI model is adapted to alert virtual nurses when it identifies potential sepsis risk factors, such as elevated inflammation markers and an existing infection source. The alerted virtual nurse then conducts a swift and comprehensive patient assessment, and based on the evaluation, initiates appropriate sepsis interventions.

In yet another aspect, the system integrates Augmented Reality (AR) technology to provide virtual nurses with an immersive, real-time view of the patient's condition. With AR, virtual nurses interact with digital patient data superimposed onto the live camera feed, offering an enhanced, holistic patient assessment that closely mirrors on-site patient care.

Upon determining a patient to be septic, the virtual nurse can leverage AI-generated intervention suggestions to initiate a rapid and effective response. For example, this may involve alerting the on-site medical team, modifying the patient's care plan, or triggering sepsis treatment protocols. The virtual nurse system is engineered to seamlessly interface with various hospital systems and patient monitoring devices, ensuring a consistent flow of patient data and facilitating real-time, collaborative decision-making among healthcare professionals.

By incorporating remote virtual nurses, the system expands the scope of the sepsis detection process, enabling 24/7 patient monitoring and immediate intervention. This enhancement significantly reduces the delay between sepsis onset and treatment, improving patient outcomes. Furthermore, this democratizes access to quality care, extending the reach of expert nursing to remote or under-resourced healthcare facilities. Accordingly, the system and method provides AI-aided virtual nursing (capability), an approach that combines human nursing expertise with advanced AI capabilities in patient care.

Among other things, the incorporation of these features into preferred system and methods may work to reduce healthcare professionals' workload, enhance continuous monitoring, and allow swift interventions, thereby enhancing patient outcomes acting as a backstop to avoid any fallouts. For instance, if a patient has an elevated heart rate and fever, the virtual nurse can alert the medical team even before the AI system determines a high risk of sepsis. This ensures a dual layer of protection: the AI's predictive capabilities and the virtual nurse's monitoring (and/or human feedback loop).

In alternative or further embodiments, the systems and methods described above may be equipped with various means to accommodate different data sources, integration methods, and disease detection algorithms. For example, the AI model may be expanded to encompass additional machine learning techniques or optimized for specific patient populations or disease conditions. Additionally, the system can be customized to assimilate progressions in healthcare technology, such as new patient monitoring devices or EHR functionalities.

### Miscellaneous Features

In yet another aspect, the system and method provide feedback to healthcare professionals through various means. The method may utilize, for example, visual representations of patient screening statuses and alerts or notifications related to sepsis risk. This feedback mechanism allows healthcare professionals to monitor and track patient progress and respond promptly to potential sepsis cases

In another aspect, the system and method according to the present disclosure incorporates “Personalized Medicine.” That is the AI model is utilized to identify patient-specific risk factors and customize the sepsis screening tool to each patient's unique needs. or example, cancer patients exhibit heightened susceptibility to sepsis due to factors like immunosuppression from treatments, the malignancy itself, and invasive procedures. Distinguishing sepsis symptoms from cancer or treatment side effects can be challenging, leading to potential delays in diagnosis. Outcomes in this group are often less favorable, with higher mortality rates and complications arising from antibiotic resistance and therapeutic interactions. Sepsis episodes can disrupt cancer treatments, necessitating modifications or delays which could influence prognosis. By phenotyping sepsis in cancer patients more targeted treatment can be achieved. Healthcare professionals gain deeper insight into individual patient vulnerabilities and tailor treatment plans accordingly.

Healthcare professionals gain deeper insight into individual patient vulnerabilities and tailor treatment plans accordingly For example, healthcare professional may stop giving antibiotics earlier and may start chemotherapy after sepsis earlier. Currently, there is a mandatory six-week time-out to start chemotherapy post sepsis. With more accurate sepsis detection, particularly, in identifying patient phenotypes, in oncological patients, chemotherapy may be re-started earlier with increased confidence in being able to detect new sepsis onset.

The sepsis detection system is preferably equipped with an intuitive and user-centric interface, enabling healthcare professionals to easily access and interact with the system. Advanced visualization techniques, voice commands, or mobile applications for remote access and monitoring can be incorporated to enhance the overall user experience.

In further embodiments, the system and method feature automated treatment recommendations, wherein the AI model is expanded to recommend appropriate treatment options based on the patient's condition, medical history, and individual characteristics. By providing treatment recommendations, the system further amplifies patient outcomes and support healthcare professionals in making prudent decisions more efficiently.

In alternative embodiments, the system and method feature and/or utilize integrated Telemedicine Platforms in the management of a patient and/or the detection of sepsis. A suitable sepsis detection system may be integrated with telemedicine platforms to facilitate remote monitoring and consultations. In this way, healthcare professionals may monitor sepsis risk in patients from a distance and provide prompt interventions, as deemed necessary.

In preferred embodiments, the system and method feature Real-time Prediction and Alert modes. The AI model is optimized to provide real-time predictions and alerts to healthcare professionals as new data becomes available. This enables quicker detection and intervention in sepsis cases.

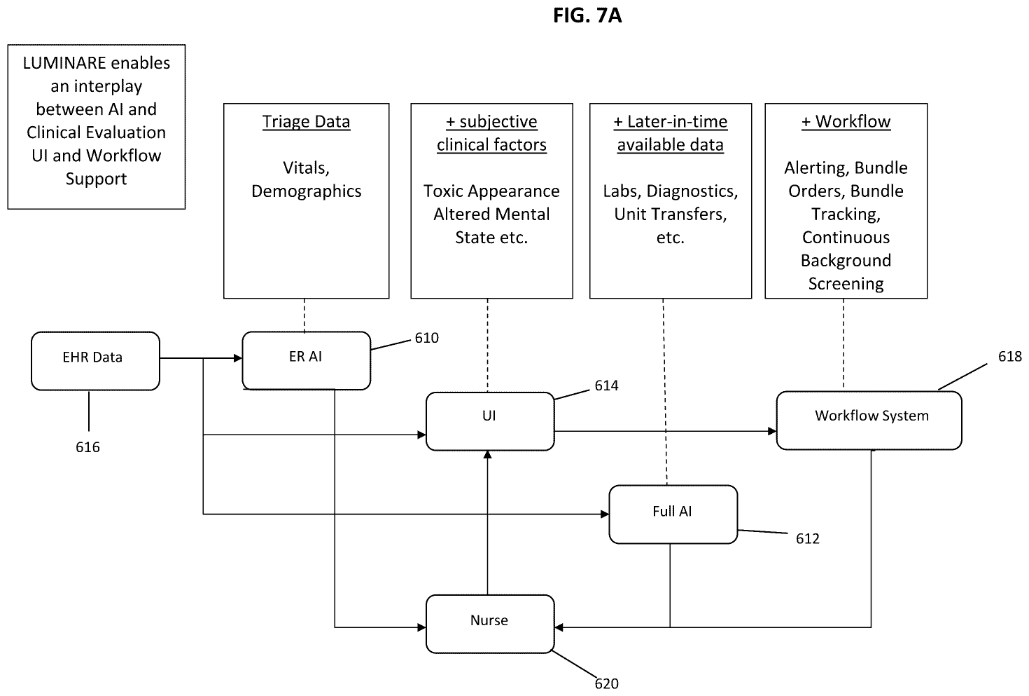

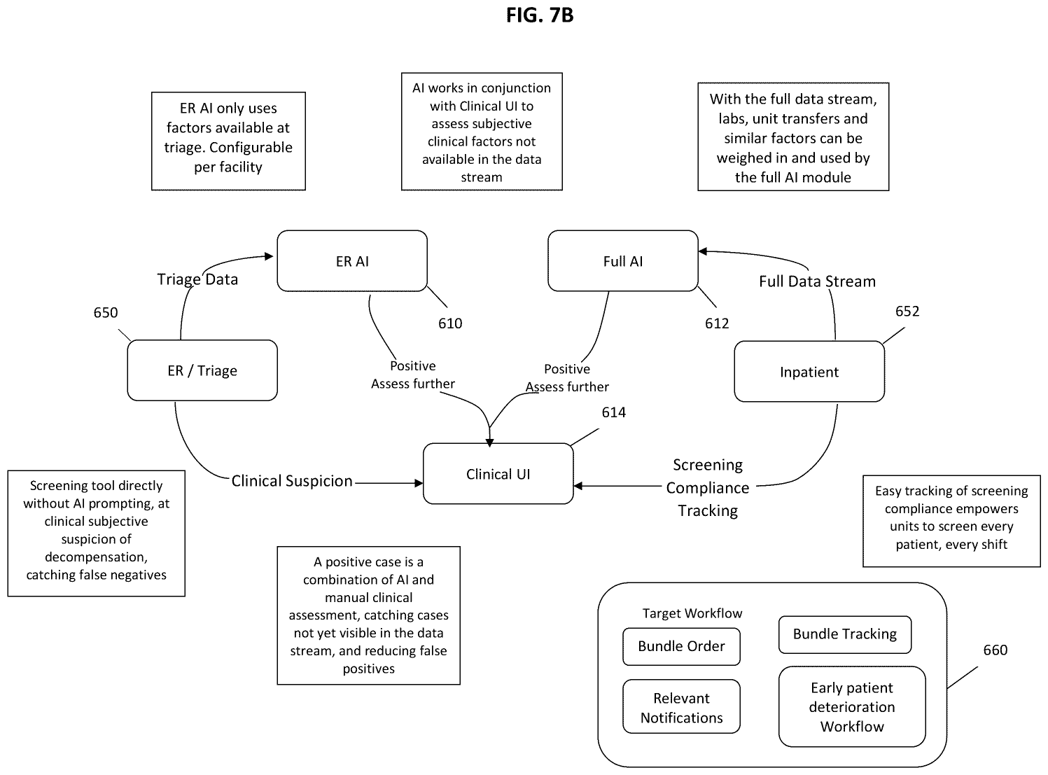

FIGS. 7A and 7B illustrates integration of the systems and methods of assessing sepsis risk within a healthcare environment, and further, into and within the critical workflows. These FIGURES also show how the system enables efficient and effective interplay between use of the AI Model and clinical evaluation. The disclosed system provides a ready means for risk assessment, including continuous monitoring and alerts, and, thereby, provides workflow support. In this context, the system, and its utilization, may be characterized as being of two parts—an ER AI 610 and Full AI 612.

A patient admitted in the ER is seen by medical personnel and, typically, triage data is collected. Triage data include vitals, and demographics. The patient is introduced into the system as the ER AI 610 receives the triage data collected in the ER and any other data compiled in the EHR data 616 (if different). Medical personnel, i.e., Nurse 620, interfaces with the system via User Interface (UI) 614. The Nurse 620 receives information and alerts from the ER AI, and may feed additional patient data into the AI. This data may include such “subjective” clinical factors as appearance (e.g., toxic appearance), mental state (e.g., altered), etc.

Post ER admission, the patient may be admitted into the hospital or clinic for further care. At this point, Full AI 612 may be said to be engaged with the patient's care, and interact with the Nurse 620. At this stage, Full AI may receive additional “later-in-time” data from such sources as lab work, diagnostics, unit transfers.

In exemplary systems, the workflow system 618 has an inbuilt auditing system that is measuring expected behavior against real world behavior. If a patient requires labs to be ordered, for example, but labs were not ordered, the system will automatically identify who is on the patient's care team from the EHR data cross reference to the persons preferred contact method (text, email, phone call etc.) and then send a targeted notification with the patient's details and the expected behavior. If the expected step is not performed, the system will then escalate to the supervisor of the care team. If the behavior expected is still not obtained, it will escalate to the supervisor of the unit until ultimately it will escalate to a sepsis team responsible for the institution. The system will also provide metrics and reports on benchmarks such as time to treatment, time to notification response that can be configurable based on the organization.

The flow chart of FIG. 6B illustrates a general workflow in the healthcare environment, as preferably aided by the systems and methods of the disclosure, particularly a system for assessing sepsis risk in a patient aided by an AI Engine or Model. This discussion highlights the two inflows for patient care-ER/Triage 650 and Inpatient 652. As described above, the Inpatient workflow concerns the longer or later time care of a patient in the clinic. The patient admitted in the ER may advance to the Inpatient environment.

As mentioned above, utilization of the preferred system and AI Model may be characterized in two parts-ER AI 610 and Full AI 612. Configurable per facility, the ER AI receives and uses data or factors collected at ER 650 during. The ER AI uses data or factors available at triage, and delivers a sepsis risk assessment. In the example, this is shown as a “positive” sepsis assessment (it could also be a degree of risk or score) and is delivered to the Clinical UI 614, where it is accessible to selected personnel and systems. At this point, a manual assessment may also be made (e.g., to confirm sepsis risk or risk factors). AI works in collaboration with Clinical UI 614 to assess subjective clinical factors that are not otherwise available in the data stream (available to the system or clinic). In the example, a combination of AI assessment and clinical assessment confirms a positive case of sepsis. The system is utilized, in this way, to catch sepsis that may not be apparent from the current data stream and also tom reduce the number or likelihood of false positives. Notably, the availability of AI does not preclude the clinic from identifying sepsis risk in a patient at the ER. As shown, the medical personnel may rely on subjective suspicion, using a screening device to prompt and confirm a positive case, thereby catching or reducing the likelihood of false negatives.

On the Inpatient node 652, a “full” data stream is available to the Full AI Model 612 These may include laboratory results, unit transfers, and similar factors. In the example, the system and AI makes a positive assessment, and then, Clinical UI 614 assesses further. Together, Full AI 612 and clinical UI 614 confirms a positive sepsis case. With a positive assessment, the system then sends guidance to or aids in the normal or target workflows or clinic tasks, including prompting or enhancing Bundled Orders, Notifications, Bundle Tracking, Early Patient Deterioration. In preferred applications, the system provides, as outputs, patient Sepsis Risk, Phenotypes, as well as action items.

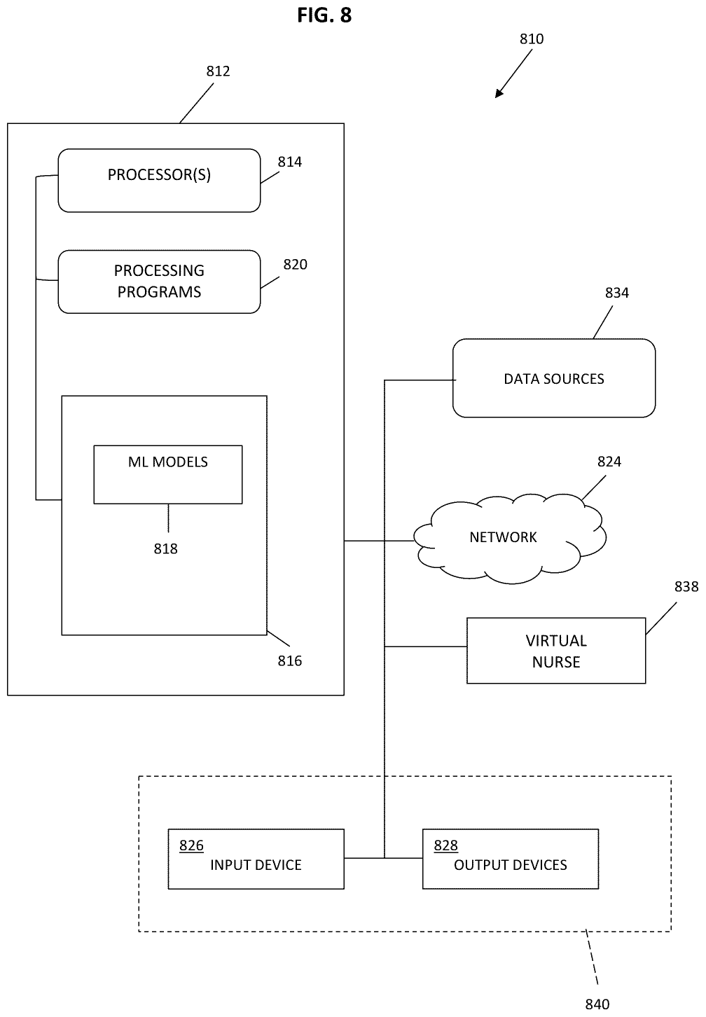

The simplified diagram of FIG. 8 provides a representation of a computing network and system and the physical and functional relationships among its components. The networked system 810 is show configured to perform the processes discussed above, including performing a medical condition risk assessment of a patient that is the object of medical care and monitoring, as discussed above. The system 810 is further configured for supporting and deploying an AI Model 810 and for supervised and continuous learning, including system and human feedback. It should be noted that the system 810 provides just one configuration and network of components suitable for supporting the system and implementing the methods and processes described above. To this end, the system 810 includes processing and storage capabilities provided in this logical representation by computing system 812, and one or more processors 814 and memory storage 816 supported therein or thereby. The processor 814 may be served by a programmable logic device or CPU, or multiples or clusters of such devices, capable of executing instructions and mathematical processing. In this exemplary system 810, the system 812 contains (or configured in communication with) the various programs and routines described above for operating and training the AI Model, which are cumulatively represented as Learning Models 818. The system 812 may also support, store and execute programs and routines, represented as a programs module 820, embodying computer executable instructions to perform the processes discussed above, including the conveying alerts, displays, and other functions and features of the system.

The computing system 812 may be connected to other computers and other system components in a distributed cloud network 824 and the internet. Further, the computing system 812 may comprise of multiple, interconnected computing systems on the network 824, and the desired programs, routines, modules, or models may reside across the multiple systems and deployed and operated therefrom, as will be generally known to one familiar with the art. Computer readable or executable instructions as described herein may be stored in memory storage, in one or more combinations, across the system and network, and/or stored and downloaded from media generally available to software services providers an health care institutions.

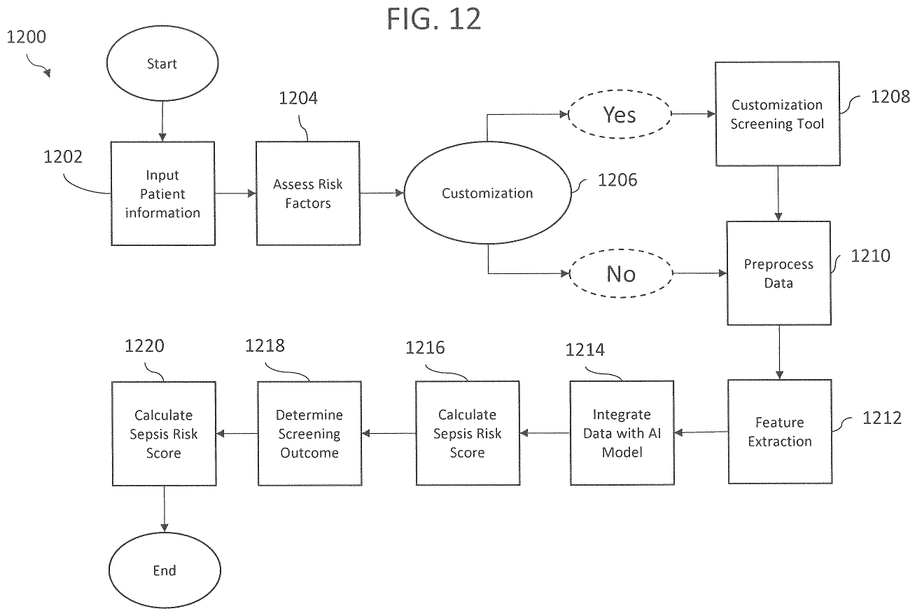

The system 810 may be configured with generally known and available network hardware and interface devices, which facilitate communications between system components. The system configuration of FIG. 12 is shown so as highlight an advantageous application in a clinic setting or healthcare facility 840, which entails medical personnel attending to, in real time, an admitted patient in collaboration with the system 812 deploying the AI Model(s) 818 to assess sepsis risk. Meanwhile, with ongoing interactions between the hospital setting 840 and the computing system 812 during management of the patient care, the Model 818 continually learns as it receives input data and, further, receives human feedback. To this end, the system 812 is shown configured with and utilizing multiple input devices 826 and output devices 828. In this example, the input and output devices 826, 828 are shown residing in the hospital or clinic environment 840, and thus, accessible and operable by the attending medical personnel. As discussed herein, the input devices 826 may comprise a patient screening tool and patient monitoring devices (e.g., monitoring heart rate, respiratory rate, etc.). Further, the screening tool, which is actually both hardware and software, may be used any medical personnel to input observations, actions taken, and other information to communicate to the AI Model 818. Moreover, the screening tool is preferably customized to prompt and query medical personnel so as to allow retrieval of such information, and in such format, that facilitates the risk assessment and also continuous training of the AI Model 818.

The screening device is preferably an I/O device, providing also the primary output device 828 through which the computing system 812 may convey its sepsis risk assessment. As well, standard computing devices such as personal computers, handheld CPU bearing devices, smart phones, tablets, and the like may be available to medical personnel to communicate with the system 812. Also, the system 810 may integrate common alarms and access to monitoring device software to communicate information (sepsis risk assessment), and in this regard, such electronic devices common to most healthcare facilities function as output devices 828 suitable for preferred systems and methods according to the present disclosure. Upon receiving and processing patient data on admission (or post admission monitoring), the computing system 812 may determine the presence of or high risk of sepsis and communicate an alert to medical personnel via the output devices 828, or, determine a low or no risk of sepsis, or no change in status. In any case, such additional information-regarding the patient and the treatment—is stored by the system and incorporated into the population of data sets accessible by the AI Model.

To further illustrate advantageous features of preferred systems and methods of assessing sepsis through deployment of an AI Model or engine, and the continual training of the Model, the system 810 is shown equipped with a database or data source(s) 834. The data source(s) may be one or multiple databases configured within the network 824 and in communication with the computing system 812. As suggested in examples described above, the data sources may include data bases of historical data external of the computing system 812 and the hospital environment 840, but accessible to the computing system 812 and AI Model 818. Further, the database(s) 834 communicates with computing system 812 and the input and output devices 826, 828. The data source 834 serves to continually store data through the duration of a patient's care and beyond to all patients, whether a patient develops sepsis or not, and regardless of the risk assessment conducted by the system or medical personnel. In preferred applications, the data source comprises an Electronic Medical Records database, which may be common to a healthcare facility or system of healthcare facilities. The Data Source 834 may also include a database external of the healthcare system such as de-identified database of external patient records.

The system also includes a virtual nurse module 838 as described herein in more detail. The virtual nurse module 838 preferably has I/O device capabilities that allow it to receive sepsis risk assessment output and also, allow it to input patient data, such as observations, into the system 812. As represented in FIG. 8, the virtual nurse module 838 may be external of the clinic environment but configured in communication therewith via the network 824

### Generating Core Sepsis Phenotypes for AI Model

In accordance with a further aspect of the present disclosure, systems and methods of assessing risk generate core sepsis phenotypes from large populations of patient data (or other sepsis related data), and utilizes the data distributions in the systems and methods discussed above. The data distribution of phenotypes may be used in training the AI Model and in the method of assessing sepsis risk in patient. As will become evident from the description below, this data distribution of phenotypes has further utility in patient management and, particularly, sepsis patient management.

Further, sepsis is a heterogenous condition and classically the approach to sepsis diagnosis entails classifying the disease into only three conditions sepsis, severe sepsis or septic shock. In accordance with the present application, at least seven core phenotypes are identified in the general patient populations each with three subgroups, with additional phenotypes in special populations like patients with cancer or other comorbidities.

Using software to identify phenotypes is essential as this cannot be done by even an expert human due to the vast number of permutations and combinations of different factors that comprise a phenotype categories including vital signs, source of infection, signs of end-organ damage, pre-existing comorbidities, age, sex, race, ethnicity, geographic location and so on. Additionally in the future adding microbiome and genetic data when commercially available will add further difficulty.

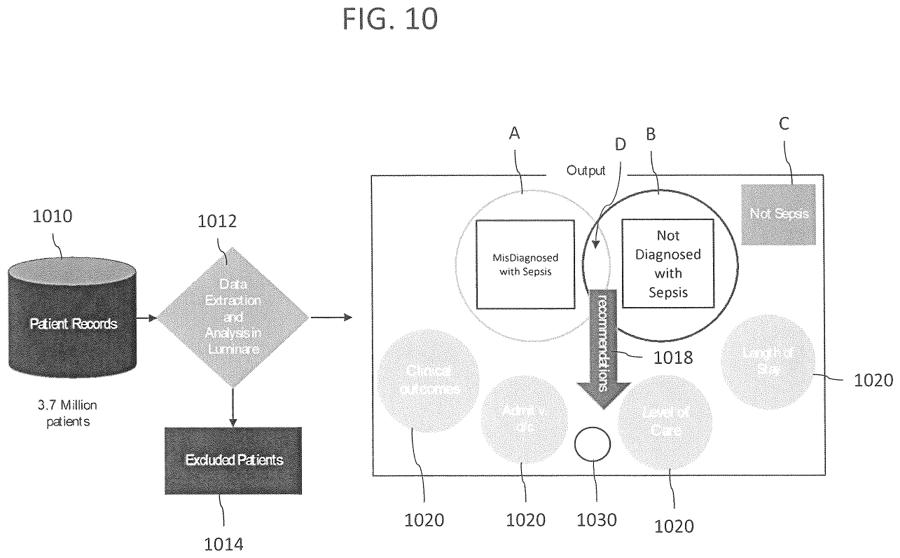

For present purposes, a core sepsis phenotype is based on or generated from observable traits(s) identified in a large distribution of sepsis patient data. FIGS. 9 and 10 illustrate how the phenotypes are generated from data sources of patient information. Initially, a large population of data is extracted from a set of varied data sources (910). In the example of FIG. 9, the exemplary data sources include HL7 data D1, HIR data D2, and User Input D3. Core sepsis phenotypes are identified through use of an expert system and machine learning techniques applied to the data. As FIG. 9 shows, multiple AI modules are deployed and work together to produce a screening outcome that is a “slice in time” of a patient's data. The slice in time is then compared to previously established sepsis phenotype classification system that then determines what the patient's current phenotype is. This is important as the classification system of phenotypes changes in order to accommodate changing populations and data drift.

As has been discussed herein, the expert system 922 in FIG. 9 may employ algorithms to output sepsis risk assessment (and in further or mature embodiments, wherein the system of phenotypes is established, a patient assignment of phenotypes). This assessment and other information provide data feed into the ML model 924. In any event, and returning to FIG. 9, the expert system 922 and model 924 process data and transform it (912) into screening outcomes 926 (sepsis risk, specific outcomes, SIRS etc.). From these outcomes, the system generates classifications 928, and from there, phenotypes 930 are derived. The flowchart depicts not just a one-time data processing sequence but a cyclical, evaluative process where data is continuously fed back into the system via the Expert System and the ML model. This setup allows for the ongoing enhancement of the system's ability to detect, classify, and manage sepsis risks more effectively.

Notably, in the handling of sepsis data, the system advantageously integrates an ML model, as shown in FIG. 9, which employs reinforcement learning from human feedback (RLHF) (as discussed above). This ML model complements the existing expert system (which may be implemented via operation of the screening tool) by using the expert systems outputs as inputs for further refinement of its algorithms and learning. Thus, the ML model continuously updates its understanding and prediction capabilities based on new data. This helps to address issues like concept drift, which proves advantageous given the dynamic nature of sepsis presentations in different patient populations. This ongoing learning process helps in generating accurate classifications and continually updated phenotypes, ensuring that the AI's outputs remain relevant and accurate over time and as data characteristics change.

More specifically, the expert system processes initial data from sepsis screenings to determine potential sepsis outcomes. This output is then fed into the ML model as critical feedback. The ML model uses this information to refine its predictive algorithms and generate more accurate classifications and phenotypes. Thus, the interaction between the expert System and the ML Model exemplifies a feedback loop where initial screening outcomes are enhanced through machine learning, providing a layered approach to data evaluation and sepsis detection. It should be noted that processes shown in FIG. 9 may be initiated with a sufficient volume of new data or as different data sources become available. The result will be confirmation of the current set of phenotypes and/or generation of new phenotypes (and subtypes). Referring now to FIG. 10, the block diagram provides, in basic form, process preferably employed in generating (at least initially) a system of phenotypes. The process first entails providing access to a large population of patient records 1 (010), and then extracting and performing an analysis of the data sets (1012). The system will necessarily extract certain patient records (1014). The patient records will include large array of patient related data throughout the relevant period, treatment, and treatment outcomes, responses, and diagnoses (including whether sepsis subsequently presented in the patient (e.g., after diagnoses). The output of the subprocess (1016) is provided as box 1016 in FIG. 10, which includes patients (i.e., patient records) who were diagnosed with sepsis (A), patients with sepsis (B), and the remaining patients that did not present with sepsis and were not diagnosed as such (C). The portion D represents patients with sepsis and who were correctly diagnosed with sepsis. To the left of D i.e., the part of A not included in D, are those patients that were misdiagnosed (incorrectly diagnosed with sepsis) but did not present as such. Associated with this portion D are the treatment recommendations.

The system, and AI model is then deployed (1018) to process each sepsis case (from A and B sets) and provide as output the treatment recommendations. The treatment recommendations are indicated as outputs 1020 in FIG. 10. The AI model also outputs an initial set of phenotypes 1030, as discussed herein, on analysis of the patient records, including outcome. The system discovers relevant correlations between the features or trait among patient (records) and, particularly, patient outcomes (sepsis) to establish a system of phenotypes, the details and use of which are discussed below. In future and further iterations of the process, new data will be incorporated and analyzed confirm, adjust, and or update (collectively, calibrate or recalibrate) the system, the AI Model, and/or the system of phenotypes (and subtypes). Moreover, as new or current patient data is processed, and the system and model is used to assign a particular phenotype to a patient, the system assesses correlations that may indicate additional factors that may be extracted to add additional functionality to the system as a whole. This explainability would be leveraged to improve existing phenotypes and possibly to create entirely new groupings of patient classes.

For example, in a test case, data is accessed or obtained from geographically heterogenous populations from sites across the United States, Brazil and Israel. The core phenotypes represent distinct patterns of clinical characteristics, laboratory findings, and disease progression observed in patients with sepsis. Additionally, more granular subtypes within each core phenotype account for variations due to factors such as age, comorbidities, and infection sources. The present AI model leverages these phenotype definitions to more accurately classify patients and tailor sepsis risk assessments based on their specific clinical presentation. As new data become available, the phenotype definitions may be iteratively refined, allowing the system to continuously improve its predictive capabilities.

### Sepsis Phenotype Development

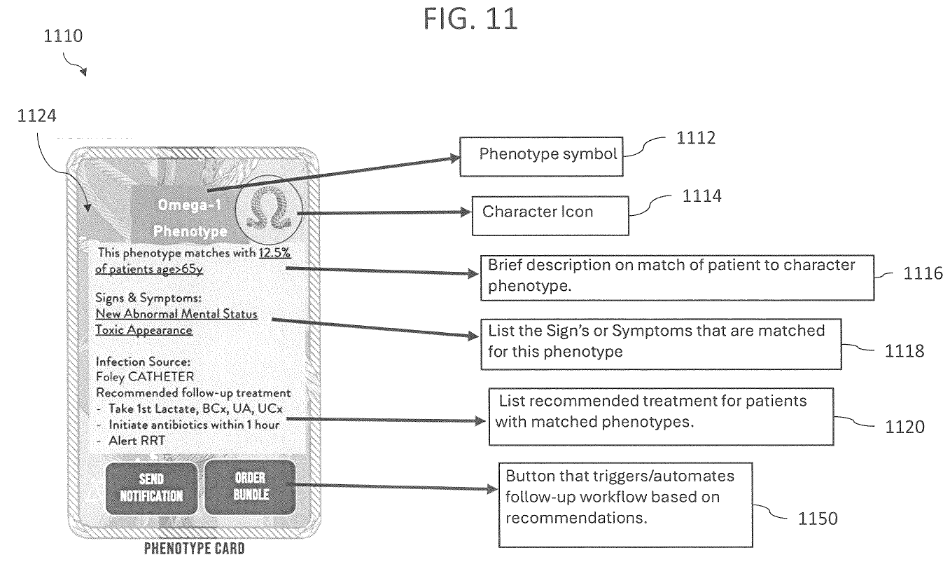

A further aspect of the disclosed sepsis detection system and method is the data-driven development of core sepsis phenotypes and subtypes from a large real-world patient dataset. As depicted in the examples of FIGS. 10 and 11, this process leverages a dataset of several millions of patient records obtained. Through advanced data extraction pipelines utilizing the system, relevant clinical data is extracted from this massive dataset, including patient demographics, diagnoses, laboratory results, vital signs, and other pertinent information. This extracted data undergoes rigorous preprocessing and cleaning procedures to ensure data quality and consistency. Subsequently, machine learning algorithms are applied to identify distinct patterns and clusters within the patient data, representing different phenotypic expressions of sepsis.

To illustrate, the core sepsis phenotypes, labeled as ALPHA, BETA, GAMMA, EPSILON, PSI, and ZERO in Table 1, capture the most prominent and clinically significant variations in how sepsis manifests across different patient subgroups. Within each core phenotype, the system and method further delineates more granular subtypes based on factors such as age, comorbidities, infection sources, and other relevant patient characteristics. This level of granularity enables the AI model to provide highly personalized risk assessments and detection strategies tailored to each individual patient's unique clinical profile.

Table 1 provides an exemplary lists of various phenotypes for a medical condition or disease condition such as sepsis, their respective descriptions, and the combinations of clinical indicators used to characterize each phenotype. The table incorporates abbreviations for common clinical parameters such as temperature (Temp), heart rate (HR), respiratory rate (RR), blood pressure (BP), white blood cell count (WBC), presence of immature neutrophils (Bands), acute mental status change (AMS), and toxic appearance (Tox. App.).

TABLE 1
Base Sepsis Phenotypes for Adults

| Sepsis | |
|---|---|
| Phenotypes | Description |
| α-1 | Abnormal Temperature + any one or more of the SIRS |
| | factors + one or more SOI |
| α-2 | α-1 with EOD |
| α-3 | α-2 with Septic Shock |
| β-1 | normal Temperature + any two or more of the SIRS |
| | factors + one or more SOI |
| β-2 | β-1 with EOD |
| β-3 | β-2 with Septic Shock |
| γ-1 | Toxic Appearance + one or more SOI |
| γ-2 | γ-1 with EOD |
| γ-3 | γ-2 with Septic Shock |
| Zero-1 | any two or more of the SIRS factors |
| | OR toxic appearance (by itself) + |
| | No documented reason for source of infection |
| Zero-2 | Zero-1 with EOD |
| Zero-3 | Zero-2 with Septic Shock |
| Ψ-1 | Abnormal BP with AMS + one or more SOI |
| Ψ-2 | Ψ-1 with EOD |
| Ψ-3 | Ψ-2 with Septic Shock |
| Ω-1a | Abnormal AMS, normal RR and one or more SOI and/ |
| | or EOD |
| Ω-1b | Abnormal AMS and RR with one or more SOI and/or EOD |
| Ω-2 | Abnormal RR, AMS with one or more SOI and EOD |
| Ω-3 | Ω-2 with Septic Shock |

AMS-altered mental status
EOD-end organ dysfunction
RR-respiratory rate
SOI-source of infection
Patient of the following groups
Table 2 provides examples of phenotypes of non-adult populations

The classification system is different from adults as age is the strongest determinant factor that outweighs all other factors as a common denominator for these patients. Age also determines the patient's immunization status, exposure to various microorganisms socially and other socioeconomic determinants of health.
Neonatal Phenotypes NPe
NP—Subgroup 1a—Newborn 0 d-7 d with identified source of infection 1b—no identified source of infection
NP-Subgroup 2—Neonate 8 d-28 d—1a
Pediatric phenotypes-PP
PP-Subgroup 1 Infant 31 d-1 y
PP-Subgroup 2 Toddler/Preschool >1 yr-5 y
PP-Subgroup 3 School age >5 yr-12 y
PP-Subgroup 4 Adolescent >12 yr-18 yr

TABLE 2
Base Sepsis Phenotypes for Neonates and Pediatric Patients

| Sepsis | |
|---|---|
| Pheno- | |
| types | Description |
| NP-1a | Neonate patients ages 0 d-7 d with identified source of |
| | infection |
| NP-1b | Neonate patients ages 0 d-7 d with no identified source of |
| | infection |
| NP-2a | Neonate patients age 8 d-28 d with identified source of |
| | infection |
| NP-2b | Neonate patients age 8 d-28 d with no identified source of |
| | infection |
| PP - 1a | Infant patients ages 31 d-1 yr with identified source of |
| | infection |
| PP-1b | Infant patients ages 31 d-1 yr with no identified source of |
| | infection |
| PP-2a | Toddler/Preschool patients ages between 1 yr to 5 yr with |
| | identified source of infection |
| PP-2b | Toddler/Preschool patients ages between 1 yr to 5 yr with no |
| | identified source of infection |
| PP-3a | School age patients ages between 5 yr to 12 yr with identified |
| | source of infection |
| PP-3b | School age patients ages between 5 yr to 12 yr with no identified |
| | source of infection |
| PP-4a | Adolescent patients ages 12 yr-18 yr with identified source of |
| | infection |
| PP-4b | Adolescent patients ages 12 yr-18 yr with no identified source of |
| | infection |

As shown in Table 1, preferred sepsis phenotypes span a wide range of clinical presentations, from abnormal temperature combined with other systemic inflammatory response syndrome (SIRS) factors (e.g., α-1, α-2, α-3), to more subtle manifestations involving altered mental status or respiratory rate changes (e.g., Ω-1a, Ω-1b, Ω-2, Ω-3). The phenotypes also account for the presence or absence of a documented source of infection (SOI) and the development of end-organ dysfunction (EOD) or septic shock.

The “Zero” phenotypes (Zero-1, Zero-2, Zero-3) represent a unique group of patients exhibiting SIRS (systemic inflammatory response syndrome) factors or toxic appearance without a clearly identifiable source of infection at the initial assessment. These phenotypes are transient, as patients may either progress to a more definitive sepsis phenotype if an infection source is subsequently identified or be excluded from the sepsis diagnosis if no source is found.

### Sepsis Phenotype Visualization and Clinical Decision Support

To facilitate rapid recognition and clinical decision-making based on the identified sepsis phenotypes, the system preferably employs intuitive visual representations in the form of m “Sepsis Phenotype Cards” 1110 as shown in FIG. 11. Each card 1110 corresponds to a specific core phenotype (e.g., ALPHA, BETA) or subtype, presenting key characteristics and recommendations tailored to that particular sepsis manifestation.

Referring to FIG. 11, a phenotype card 1110 is shown with a front display 1124 for communicating information regarding the patient, the phenotype, and related information. For convenience and durability, the card may be made thin and credit-card size. More preferably, the card 1110 is electronic with a front display 1124 that includes and supports communication means for triggering messages or alerts for the clinician. Preferred communication means may include email, text and telephony via existing conventional technology. The depicted card 1110 and its display 1124 is preferably configured to present an educational prompt to a bedside clinician, for example, and advise on the indicated phenotype, its prevalence and recommended clinical actions. The card 1110 in FIG. 11 includes a button 1150 that indicates it will “Initiate Recommended Treatments,” This trigger preferably triggers or automates follow-up workflow based on recommendations. For example, the trigger may be used to initiate a nurse driven foley catheter removal protocol or recommend changing out an ageing central venous catheter for a new catheter over a guidewire.

The card 1110 is preferably equipped with an electronic display 1124. The card 1110 displays the phenotype symbol 1112 or descriptive title, which may be well associated with the particular phenotype, and a representative character icon(s) 1114 that may provide or present typical patient characteristics or presentations associated with that phenotype. The use of a character icon 1114 is intended for different levels of medical personnel and other appropriate responsible persons and designed to facilitate recognition and association of the phenotype and related treatment factors. In this respect, the card 1110 may be particularly effective in or at different levels of care and throughout the clinic environment, including at ER and Inpatient care.

The card 1110 depicted in FIG. 11 provides a brief descriptive summary or match 1116 that matches the patient's clinical picture to the corresponding phenotype, aiding in rapid pattern recognition. Preferably, the phenotype card 1110 included a listing of key signs 1118 and symptoms that are typically exhibited by patients belonging to that specific phenotype cluster. This symptomatic profile assists clinicians in swiftly identifying patients who may fit the phenotype, prompting further evaluation or intervention as necessary. More preferably, the phenotype card 1110 features on its display 1124 a listing or box 1120 providing recommended treatment guidelines or protocols tailored to the unique requirements of patients within that phenotype group. These recommendations may include specific diagnostic tests, medication regimens, monitoring strategies, or escalation protocols, all designed to optimize care delivery for patients exhibiting that particular sepsis presentation.

In an example corresponding to the card 1110 depicted in FIG. 11, the patient is assigned the phenotype with the symbol or icon for Omega” and title.” Omega-1.” Further, the card 1110 display the following display or listings 116, 118, 1120 of phenotype-derived information:
“This phenotype matches with 12.5% of patients age >65 years.” (1116) and;
Signs & Symptoms” (1118)
“-New Abnormal mental status
Toxic Appearance”
Recommended follow-up treatment (1120)
Initiate ABX, within 2 hours
Alert RRT
Administer fluids within 2 hours

The card 1112 is also equipped with two buttons or triggers 1150. One trigger 1150 effects an automated “Order Bundle”, as follow up workflow. The second trigger 1150 effects a “Send Notification” message or prompt.

The phenotype match card may be embodied in a physical, electronic device capable of receiving or transmitting signals, and preconfigured to present the selected displays. In that respect, the card may be further equipped with a frame, a display, memory storage, and/or transceiver means. Alternatively, the card may be embodied in or as a personal electronic device or varying computer processing means. These include devices such as tablets, laptops, wearables, and the like.

The flowchart of FIG. 12 illustrates a more preferred method of assessing sepsis risk that incorporates use of a system of phenotypes (as described in the preceding paragraphs). The method also preferably employs a sepsis screening tool as previously discussed. Flow charts previously described above in respect to previous embodiments, such as FIG. 3, illustrate the flow of data extraction from various sources, preprocessing, and integration into data available to the AI Model. The process illustrated in FIG. 12 may include or be combined with the same fundamental steps of these earlier-described flow charts with the inclusion of the system of phenotypes, thereby enhancing the focus on how data specificities influence the output of the AI Model. With reference to FIG. 12, process 1200 includes an input patient information step 1202. Step 1202 is an initial step in which patient data is collected. Process 1200 includes an assess risk factors step 1204, in which the initial data of step 1202 is examined to identify any sepsis risk factors. Process 1200 includes a customization step 1206. In step 1206, it is decided whether customization of the screening tool based on patient-specific factors will be performed. If the answer to step 1206 is yes, then the process 1200 includes a customize screening tool step 1208. In step 1208, the screening tool is adjusted and/or updated to better align the screening tool with identified patient-specific factors. After step 1208, the process 1200 proceeds to step 1210, discussed below. If the answer to step 1206 is no, then the process 1200 bypasses step 1208 and proceeds to a preprocess data step 1210. In step 1210, the expert system is used to preprocess the data, which includes cleaning and preparing the data by removing irrelevant or outlier data. Process 1200 includes a feature extraction step 1212. In step 1212, specific features relevant to sepsis risk are extracted based on both generic and patient-specific parameters, which are integrated after customization, if performed. Process 1200 includes an integrate data with AI Model step 1214, in which the processed and feature-enhanced data is fed into the AI model. Process 1200 includes a calculate sepsis risk score step 1216, in which the AI model analyzes the data to produce a sepsis risk score. Process 1200 incudes a determine screening outcome step 1218. In step 1218, the outcome of the screening tool, including the risk score and any phenotypes, is determined.

These and other variations of the disclosure will become apparent to one generally skilled in the relevant art provided with the present disclosure. Consequently, variations and modifications commensurate with the above teachings, and the skill and knowledge of the relevant art, are within the scope of the present disclosure. The embodiments described and illustrated herein, including algorithms, are further intended to explain best or preferred modes for practicing the disclosure, and to enable others skilled in the art to utilize the disclosure and other embodiments and with various modifications required by the particular applications or uses of the present disclosure.

The following is an exemplary listing of claims, various applications, variations, and/or embodiments contemplated by described concepts. The present disclosure describes or contemplates concepts including methods, algorithms, products, systems, user interfaces, media, articles of manufacture and other concepts discussed above, some of which are characterized by the below listing of features (presented in claim form). This list should not be considered limiting, however, as the elements or features listed below, in respect to methods, products, systems, articles, etc. may be combined with each of the other elements associated with other methods, products, systems, articles, etc. The same applies to methods and various, exemplary steps listed below. Also, the above description and the Drawings, and the claims, describe or depict other applications, variations, embodiments, and combinations of elements which may not be included below, but are contemplated as encompassed by the described concepts.

1. A system for sepsis detection using reinforcement learning from human feedback (RLHF), comprising:
a. a customized sepsis screening tool for assessing patients for sepsis, adaptable for different patient populations and specific disease conditions;
b. data integration means for seamless integration of the sepsis screening tool with electronic health records (EHRs) and patient monitoring devices;
c. an artificial intelligence (AI) model, trained using RLHF, which processes data from the sepsis screening tool, EHRs, and patient monitoring devices to generate alerts for clinicians when it suspects a screening needs to be reevaluated;
d. a granular visual input based layer that allows the healthcare professional to indicate why the AI tool's output is accurate or inaccurate through a series of pre-formatted queries;
d. feedback means for providing healthcare professionals with information related to sepsis risk and patient progress.

2. The system of claim 1, wherein the customized sepsis screening tool accounts for various factors such as age, medical history, and demographics.

3. The system of claim 1, wherein the data integration means accommodates various data formats and communication protocols to ensure compatibility with existing and future healthcare information systems.

4. The system of claim 1, wherein the AI model uses new screening outcomes as feedback to learn and improve its sepsis detection capabilities.

5. The system of claim 1, wherein the feedback means includes visual representations of patient screening statuses and alerts or notifications related to sepsis risk.

6. A method for sepsis detection using reinforcement learning from human feedback (RLHF), comprising the steps of:
a. assessing patients for sepsis using a customized sepsis screening tool;
b. integrating data from the sepsis screening tool, electronic health records (EHRs), and patient monitoring devices;
c. processing the integrated data using an artificial intelligence (AI) model continuously training with RLHF;
d. generating alerts for clinicians when the AI model suspects a screening needs to be reevaluated;
e. providing healthcare professionals with feedback related to sepsis risk and patient progress.

7. The method of claim 6, wherein the customized sepsis screening tool accounts for various factors such as age, medical history, and demographics.

8. The method of claim 6, wherein the data integration step accommodates various data formats and communication protocols to ensure compatibility with existing and future healthcare information systems.

9. The method of claim 6, wherein the AI model uses new screening outcomes as feedback to learn and improve its sepsis detection capabilities.

10. The method of claim 6, wherein the feedback provided to healthcare professionals includes visual representations of patient screening statuses and alerts or notifications related to sepsis risk.

11. A computer-readable medium containing instructions for sepsis detection using reinforcement learning from human feedback (RLHF), which when executed by a processor, cause the processor to perform the steps of:
a. assessing patients for sepsis using a customized sepsis screening tool;
b. integrating data from the sepsis screening tool, electronic health records (EHRs), and patient monitoring devices;
c. processing the integrated data using an artificial intelligence (AI) model continuously training with RLHF;
d. generating alerts for clinicians when the AI model suspects a screening needs to be reevaluated;
e. providing healthcare professionals with feedback related to sepsis risk and patient progress.

12. The system of claim 1, further comprising means for incorporating additional data sources, including but not limited to patient wearables, medical imaging, or genomics data, to improve the accuracy and adaptability of the AI model.

13. The system of claim 1, further comprising an improved user interface and experience, including advanced visualization techniques, voice commands, or mobile applications for remote access and monitoring.

14. The system of claim 1, wherein the AI model is further capable of recommending appropriate treatment options based on the patient's condition, medical history, and individual characteristics.

15. The system of claim 1, further comprising means for integration with telemedicine platforms to enable remote monitoring and consultations.

16. The system of claim 1, wherein the AI model provides real-time predictions and alerts to healthcare professionals as new data becomes available.

17. The system of claim 1, further comprising means for incorporating clinical explainability to the AI model to provide transparency and understanding of the model's predictions to healthcare professionals.

18. The system of claim 1, wherein the AI model identifies patient-specific risk factors and tailors the sepsis screening tool to each patient's unique needs.

19. A sepsis detection and intervention system as described in claim 1,
wherein the system employs remote virtual nurses to conduct proactive sepsis screenings for all patients in an inpatient unit.

20. The system of claim 19, wherein the remote virtual nurses use the patented sepsis screening tool and real-time patient data to assess sepsis risk and adapt the screening process based on individual patient characteristics and the evolving nature of their condition.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)
